(19) United States
(12) Patent Application Publication
(10) Pub. No.: US 2014/0322277 A1
(43) Pub. Date: Oct. 30, 2014

(54) PROCESS AND APPARATUS FOR OBTAINING A SOLID FORM OF MATERIAL

(71) Applicants: Dario Rea, Monterenzio (Bologna) (IT); Giorgio Manaresi, Ozzano Dell'Emilia (Bologna) (IT)

(72) Inventors: Dario Rea, Monterenzio (Bologna) (IT); Giorgio Manaresi, Ozzano Dell'Emilia (Bologna) (IT)

(73) Assignee: I.M.A. INDUSTRIA MACCHINE AUTOMATICHE S.P.A., I-40064 OZZANO DELL'EMILIA (BOLOGNA) (IT)

(21) Appl. No.: 14/114,343

(22) PCT Filed: Apr. 27, 2012
(86) PCT No.: PCT/IB12/52118

§ 371 Date: Jun. 12, 2014

(30) Foreign Application Priority Data
Apr. 29, 2011 (IT) BO2011A000233

(51) Int. Cl. B29C 53/06; A61K 9/70; B29C 43/50; B29C 53/26; B29C 43/58; B29C 43/36; A23L 1/00; B29C 53/04

(52) U.S. Cl. CPC B29C 53/06; A23L 1/0073; A61K 9/70; B29C 53/043; B29C 53/265; B29C 43/58; B29C 43/3697; B29C 43/50; A23V 2002/00; B29C 2043/5816; B29C 2043/503
USPC 424/400; 426/531; 264/287; 264/157; 425/324.1; 425/303; 425/143; 425/149

(57) ABSTRACT

In a process and an apparatus (10) for making a solid form (11), preferably of a therapeutic or pharmaceutical product, an intermediate mass (31) of material is obtained from a respective basic layer (21) in the form of a sheet or band, the intermediate mass of material being composed of a plurality of layers of material and the solid form (11) of material being obtained from the intermediate mass (31).

### TECHNICAL FIELD

This invention relates to a process and apparatus for obtaining a solid form of material or the like.

More specifically, the material is in the form of a therapeutic or pharmaceutical product, a dietary supplement and/or a food or edible product.

### BACKGROUND ART

Known in the prior art are processes and apparatuses for making a solid form, or tablet, of therapeutic or pharmaceutical product, where the solid form or tablet is obtained from a respective powder of pharmaceutical product by compression in suitable chambers and through the agency of respective piston means operating at high pressure.

These prior art processes and apparatuses, however, are made up of a large number of components and are extremely complex and thus particularly expensive and difficult to manage, especially when production is required to work several shifts a day.

Moreover, the quantity of active ingredient in each tablet must be kept under strict control to ensure it is the same in all the tablets. In these prior art processes and apparatuses, the operating methods used to do this are based on statistical considerations which are complex and expensive to implement.

Nevertheless, these operating methods still do not guarantee uniformity of results, that is to say, of tablet composition, particularly on account of variations in ambient conditions and powder composition, which cannot be promptly detected.

In practice, use of the prior art processes and apparatuses leads to frequent production shutdowns and rejection of corresponding quantities of products, in particular, when the product being made deviates excessively from production specifications.

### AIM OF THE INVENTION

This invention proposes a novel solution, alternative to the solutions known up to now, and/or, more specifically, aims to overcome one or more of the above mentioned drawbacks or problems, and/or to meet one or more of the needs mentioned or inferable from the above.

Accordingly provided is a process for obtaining a solid form of material, said material being, in particular, a therapeutic or pharmaceutical product, a dietary supplement, and/or a food or edible product, characterized in that it comprises, starting from at least one basic layer of the material in the form of a sheet or band, a step of folding the basic layer in such a way as to make a plurality of portions or laps defining an intermediate mass of material from which the solid form is obtained by separation.

Also provided is an apparatus for making a solid form of material, said material being in particular a therapeutic or pharmaceutical product, a dietary supplement, and/or a food or edible product, characterized in that it comprises folding means which, starting from at least one basic layer of the material in the form of a sheet or band, fold the at least one basic layer so as to make a plurality of laps; means which bring the plurality of laps closer together to define an intermediate mass; and means adapted to obtain a solid form from the intermediate mass by separation.

The solution thus provided is an alternative to the solutions known from the prior art, where the tablets are obtained by compression of powders.

In particular, it is possible to obtain a solid form starting from a basic layer of material which can be made using suitable, simpler methods, for example, working on the liquid phase which allows the ingredients to be distributed uniformly more easily in the basic layer.

### BRIEF DESCRIPTION OF THE DRAWINGS

These and other innovative aspects are set out in the appended claims and the technical features and advantages of the invention are also apparent from the detailed description which follows of non-limiting example embodiments of it with reference to the accompanying drawings, in which:

FIG. 1A shows a schematic perspective view of a first preferred embodiment of an apparatus according to the invention, which implements a process according to the invention;

FIG. 1B shows a perspective view of a solid form obtained with the process and apparatus according to the invention;

FIG. 1C shows a transversal cross section through the line IC-IC of FIG. 1A and illustrating in particular a section of the basic layer from which the solid form is obtained;

FIG. 1D shows a transversal cross section of an intermediate mass in the pleated condition, as it is when it feeds out of the shaping means;

FIG. 1E shows a transversal cross section through the line IE-IE of FIG. 2A and illustrates the intermediate mass with the laps in the close-together condition just before they are compressed;

FIG. 2A shows a schematic top plan view of the first preferred embodiment of the apparatus according to the invention;

FIG. 2B shows a schematic top plan view of a detail from FIG. 2A, illustrating a zone for defining and forming the solid mass;

FIG. 2C illustrates the solid form in a schematic section along a longitudinal plane L-T and shows the changes of shape of the solid form before and after the shaping and forming zone;

FIG. 2D illustrates the solid form in a schematic section along a transversal plane P-T and shows the changes of shape of the solid form before and after the shaping and forming zone;

FIG. 3 shows a schematic side view of the first preferred embodiment of the apparatus according to the invention;

FIG. 4 is a transversal cross section through the line IV-IV of FIG. 3 and shows the folding means in the first preferred embodiment of the apparatus;

FIG. 5 is a transversal cross section through the line V-V of FIG. 3 and shows the folding means in the first preferred embodiment of the apparatus;

FIG. 6 is a transversal cross section through the line VI-VI of FIG. 3 and shows the folding means in the first preferred embodiment of the apparatus;

FIG. 7 is a transversal cross section through the line VII-VII of FIG. 3 and shows the folding means in the first preferred embodiment of the apparatus;

FIG. 8 is a transversal cross section through the line VIII-VIII of FIG. 3 and shows the folding means in the first preferred embodiment of the apparatus;

FIG. 9 is a transversal cross section through the line IX-IX of FIG. 3 and shows the folding means in the first preferred embodiment of the apparatus;

FIG. 10 is a transversal cross section through the line X-X of FIG. 3 and shows the folding means in the first preferred embodiment of the apparatus;

FIG. 11 is a transversal cross section through the line XI-XI of FIG. 3 and shows the folding means in the first preferred embodiment of the apparatus;

FIG. 12 shows an enlarged schematic side view of the folding means in the first preferred embodiment of the apparatus according to the invention;

FIG. 13 shows an enlarged schematic perspective view of the compression moulding means in the first preferred embodiment of the apparatus according to the invention;

FIG. 14 is an enlarged schematic perspective view of a detail from FIG. 1A;

FIG. 15A shows a schematic perspective view of a second preferred embodiment of an apparatus according to the invention, which implements a process according to the invention;

FIG. 15B shows a schematic view of a detail from FIG. 15A, illustrating the zone for defining and forming the solid mass;

FIG. 16A shows a schematic top plan view of the second preferred embodiment of the apparatus according to the invention;

FIG. 16B shows a schematic top plan view of a detail from FIG. 16A, illustrating the zone for defining and forming the solid mass;

FIG. 17A shows a schematic side view of the second preferred embodiment of the apparatus according to the invention;

FIG. 17B shows a schematic side view of a detail from FIG. 17A, illustrating the zone for defining and forming the solid mass;

FIG. 18A shows the basic layer in a transversal cross section through the line A-A of FIG. 17A, upstream of the folding means;

FIG. 18B shows the basic layer in a transversal cross section through the line B-B of FIG. 17A;

FIG. 18C shows the second preferred embodiment of the apparatus in a transversal cross section through the line C-C of FIG. 17A;

FIG. 18D shows the second preferred embodiment of the apparatus in a transversal cross section through the line D-D of FIG. 17A;

FIG. 18E shows the second preferred embodiment of the apparatus in a transversal cross section through the line E-E of FIG. 17A;

FIG. 19A shows in a transversal cross section a second embodiment of the intermediate mass in the pleated condition, as it is when it feeds out of the shaping means;

FIG. 19B is a transversal cross section showing the second embodiment of the intermediate mass with the laps in the close-together condition just before they are compressed;

FIG. 20 is a schematic top plan view of a further preferred embodiment of the apparatus according to the invention, showing in particular the precompression means and the last stage of the folding means;

FIG. 21 is a horizontal cross section of a further embodiment of the forming means of the apparatus according to the invention;

FIGS. 22A and 22B are, respectively, a plan view and a transversal cross section of the cam means for moving the bottoms of the forming sockets used in the forming means of FIG. 20.

### DETAILED DESCRIPTION OF THE PREFERRED EMBODIMENTS OF THE INVENTION

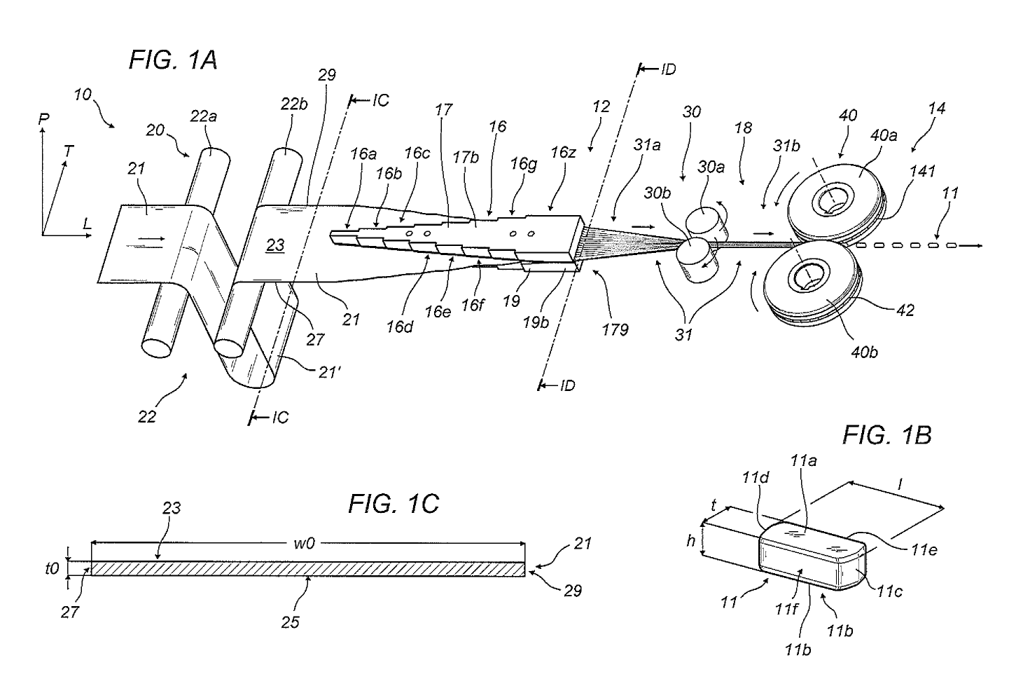

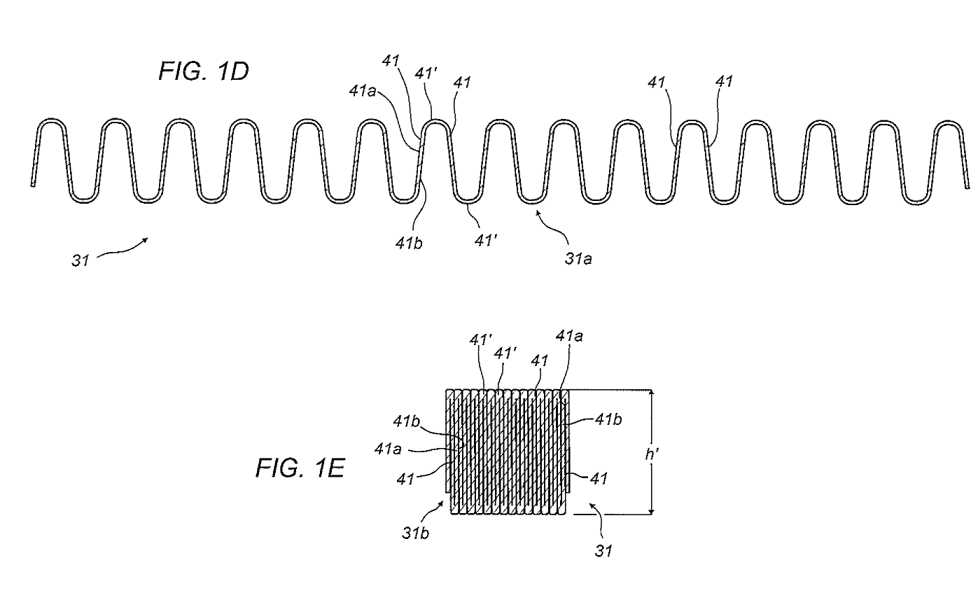

The accompanying drawings illustrate an apparatus 10 according to a first preferred embodiment of the invention and which implements a process according to the invention for making a solid form 11 (illustrated in FIG. 1B), which has respective three-dimensional dimensions, namely, a height “h”, a width “t”, and a length “l”, which are substantially of the same order of size. The solid form is made of a suitable material, preferably a therapeutic or pharmaceutical material, or a material constituting a dietary supplement, and/or a food or edible material which is intended to be ingested or taken by a person, or possibly by an animal.

For greater convenience of description, FIG. 1A shows reference axes at right angles to each other and respectively defining a longitudinal axis or direction “L”, a transversal axis or direction “T”, and a perpendicular axis or direction “P”.

As illustrated, the apparatus extends along the longitudinal direction L and has an advantageously compact configuration.

In a particularly preferred manner, the solid form 11 is obtained from a material which is in the form of a basic film, or layer, 21 made of a material which preferably contains an active pharmaceutical ingredient.

More specifically, the basic layer 21 is such that, or possesses properties such that, it can be used to make solid forms 11 with the process and apparatus of the invention without giving rise to problems.

More specifically, the basic layer 21 might contain suitable plasticizing and lubricating substances which make it suitable to undergo the operations defined by this preferred process.

More specifically, the basic film or layer might be made with or obtained from a manufacturing process known in the trade as “film casting”, which makes it possible to start from a base material in the liquid phase, thus guaranteeing a high degree of uniformity of the material, compared to other methods, in particular methods in which the raw materials used are in the solid phase.

More specifically, the solid form 11 defines a tablet, or lozenge, and might have any desired shape. In the embodiment illustrated in FIG. 1B, however, the solid form 11 has respective longitudinal faces 11a, 11b, defining the large faces of the solid form, and respective transversal faces, respectively the leading and trailing ends 11c, 11d, as well as respective lateral or perpendicular faces 11e, 11f, the transversal faces 11c, 11d and the lateral faces 11e, 11f extending between the longitudinal faces 11a, 11b.

The solid form 11 might also be of any suitable size required.

Advantageously, the process contemplates starting from a basic layer 21 of respective thickness ‘t0’ and respective width ‘w0’, and having opposite longitudinal faces 23, 25 and lateral edges 27, 29, preferably parallel, as illustrated in FIG. 1C.

The basic layer 21 is fed along a feed direction with stepping or, preferably, continuous motion.

The basic layer 21, too, may be of any size suitable for obtaining a respective solid form 11 as required.

The basic layer 21 is preferably in the form of a thin layer or film and, preferably, may be in the form of a continuous or elongate sheet or band of material, as illustrated here.

Advantageously, the process entails making an intermediate mass 31 of material starting from the basic layer 21 and composed of a plurality of layers 41 of the material.

Advantageously, according to the process, the solid form 11 is obtained by separation of the intermediate mass 31.

More specifically, as illustrated, a first intermediate mass, or first part of the intermediate mass, denoted by the reference 31a, is obtained by directly folding the basic layer 21, and a second intermediate mass, or second part of the intermediate mass, 31b, is obtained from the first intermediate mass 31a and is in the form of an elongate or continuous body as will become clearer as this description continues.

More specifically, the first intermediate mass of material 31a is, as illustrated, in the form of a layer which is pleated or comprises a plurality of layers, or pleat portions 41, 41. The term “pleat portion” is used here to mean a pair of laps, or portions, 41, 41 of the basic layer which have a fold line 41′ in common.

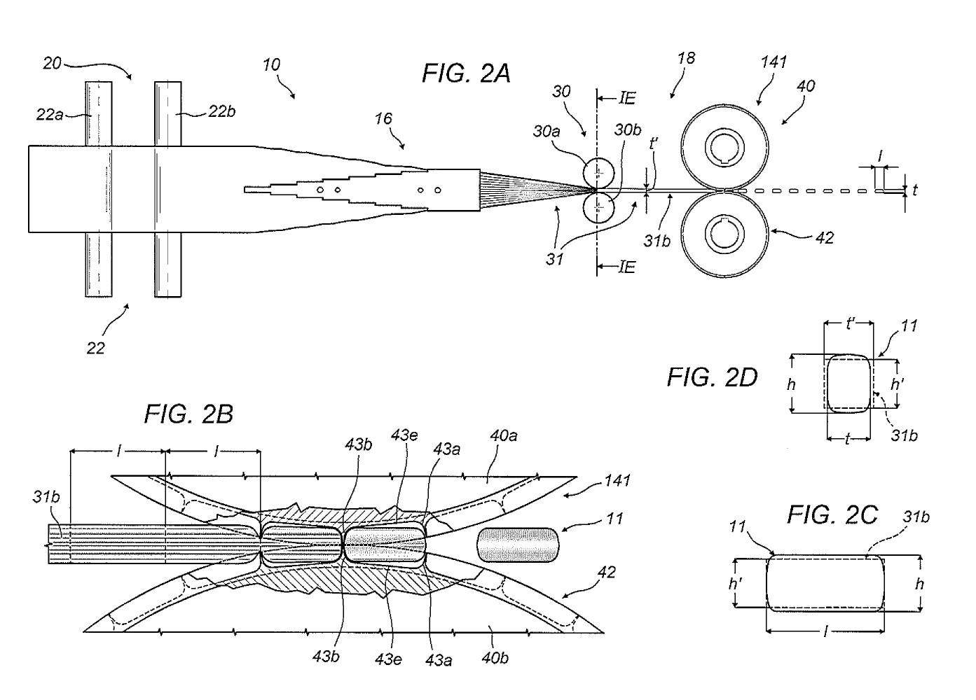

With reference to the embodiment illustrated, the intermediate mass 31 from which the solid form 11 is obtained, is defined by the second intermediate mass 31b which, more specifically, is in the form of an elongate body having a respective width t′ and a respective height h′, as may be inferred from FIGS. 2A, 2C, 2D, 3 and 14.

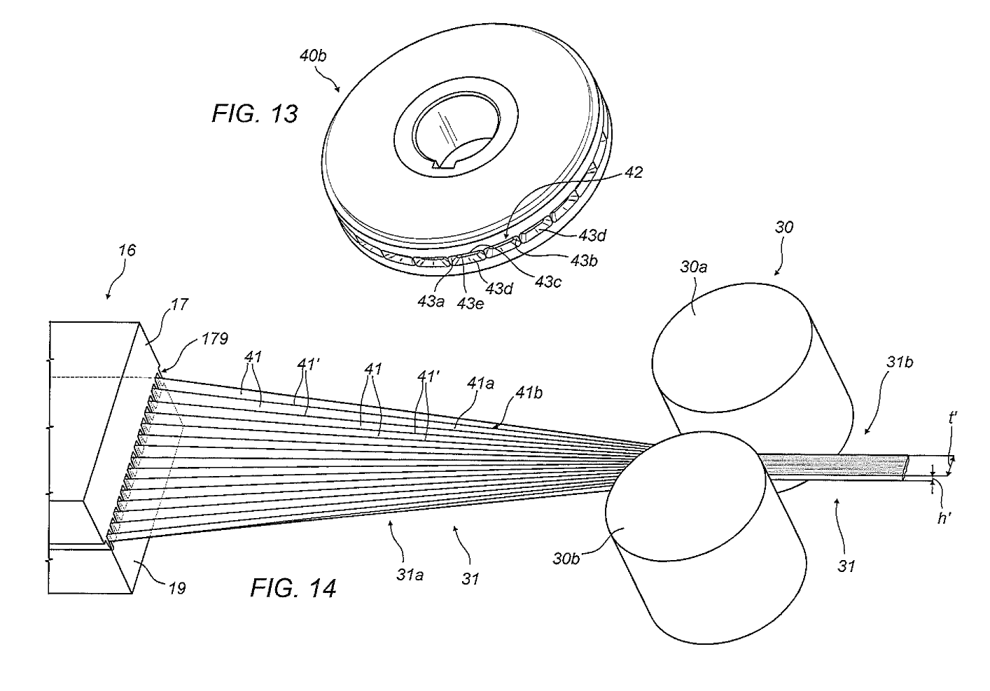

More specifically, the second intermediate mass 31b (see FIG. 14) has a substantially quadrangular cross section, and more specifically, a rectangular cross section.

Although the embodiment illustrated is the especially preferred embodiment, obtaining this solid form 11 from an intermediate portion 31 consisting only of the first intermediate mass 31a is also imaginable, however. The latter embodiment is not illustrated in the accompanying drawings.

According to the process illustrated, the second intermediate mass 31b comprises a plurality of layers 41 which are aggregated or held together. More specifically, the mass of material 31b comprises a plurality of layers 41 which adhere to each other.

For the purpose, the layers 41 of the intermediate mass 31 each have opposite outside faces 41a, 41b, as illustrated in FIG. 1D.

More specifically, the layers 41 are aggregated to each other in such a way that the outside faces 41a, 41b of one layer or lap 41 come into contact with the outside faces of a corresponding adjacent layer or lap 41, as may be inferred from FIG. 1E. As illustrated, according to the configuration of FIG. 1E, the outside faces of the layers 41 are substantially parallel to each other and perpendicular to a plane L-T defined by the basic layer 21 in a plane or unpleated condition.

As illustrated, advantageously, the intermediate mass 31 is made from a single basic layer 21. In an embodiment not illustrated, the intermediate mass 31 may be made from two or more basic layers 21 which are superposed and adherent to one another.

Also, according to the process, the layers 41 of material defining the intermediate mass 31 are defined by portions or strips, in particular longitudinal ones, of the basic layer 21.

More specifically, the layers 41 of material defining the intermediate mass 31 are defined by portions or strips of the basic layer 21 which are adjacent to each other.

The process comprises a step of folding the basic layer 21 in such a way as to define a corresponding intermediate mass 31, in particular a first intermediate mass 31a.

Advantageously, the folding step comprises extending one or more portions or laps, 41 of the basic layer 21 perpendicularly to the plane L-T defined by the basic layer 21 in the plane condition.

In other words, the purpose of folding is to shape the basic layer 21 according to a shaped profile, the shaped profile giving the basic layer 21 one or more portions, or laps, 41 which extend perpendicularly to the plane L-T defined by the basic layer 21 in the plane condition.

In practice, a basic layer 21 is provided which has a suitable transversal profile and which can define the first intermediate mass 31a, from which the solid mass 11 can be conveniently obtained according to an embodiment not illustrated in the drawings or from which it is possible, as illustrated here, to obtain the second intermediate mass 31b from which the solid form 11 can be obtained, as shown in the accompanying drawings.

More specifically, the layers 41 which define the intermediate mass 31 are obtained by suitably folding the basic layer 21.

Folding the basic layer 21 entails making in the selfsame layer 21 one or more fold lines, in particular one or more longitudinal fold lines 41′.

The fold lines 41′ are, at least until the layer of material 21 remains within respective folding means 16, parallel or substantially parallel to each other, and such as to extend along the feed direction of the basic layer 21.

Each fold line 41′ defines a respective pair of adjacent laps 41, 41, which constitute respective portions of layers of the intermediate mass 31.

More specifically, as illustrated, each lap 41 extends along a direction perpendicular, or substantially perpendicular, to the plane L-T defined by the basic layer 21 in the plane or unshaped condition.

More specifically, as illustrated, each lap 41 extends perpendicularly to, and on the opposite side of, the plane L-T defined by the basic layer 21 in the plane or unshaped condition.

Further, each lap 41 extends to the same extent on the opposite side of, and perpendicularly to, the plane L-T defined by the basic layer 21 in the plane or unshaped condition.

Advantageously, folding the basic layer 21 comprises performing a plurality of folding steps in succession, or in a plurality of successive steps, on the selfsame basic layer 21.

That way, the basic layer 21 is deformed gradually, avoiding excessive stress to the basic layer 21, and thus preventing the basic layer 21 from tearing.

In practice, as mentioned above, folding makes a plurality of pleat portions, where each pleat portion is defined by a respective first and second lap 41, 41 having a fold line 41′ in common.

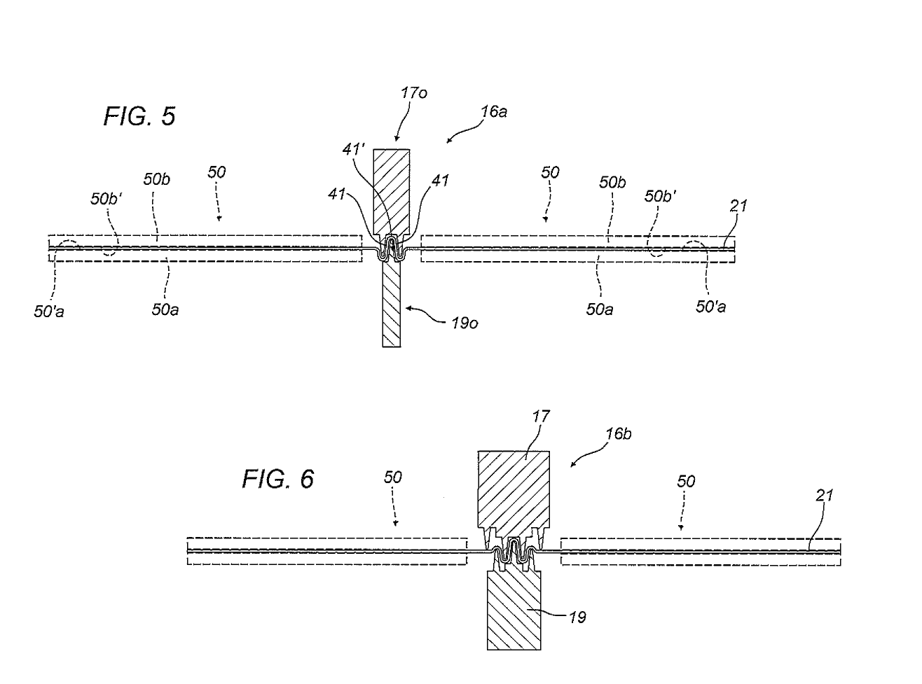

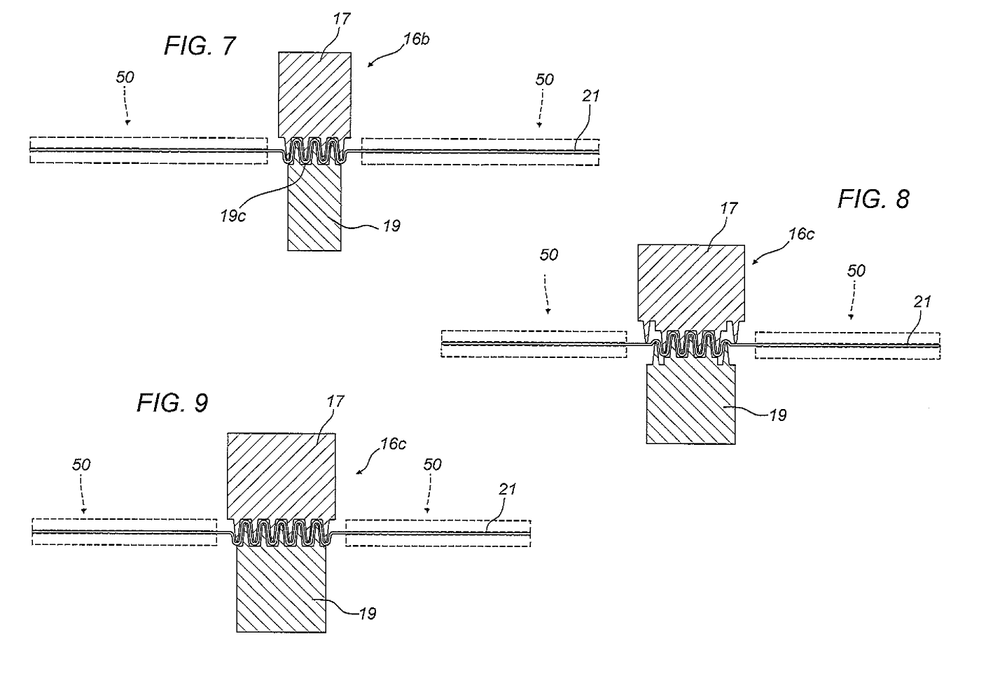

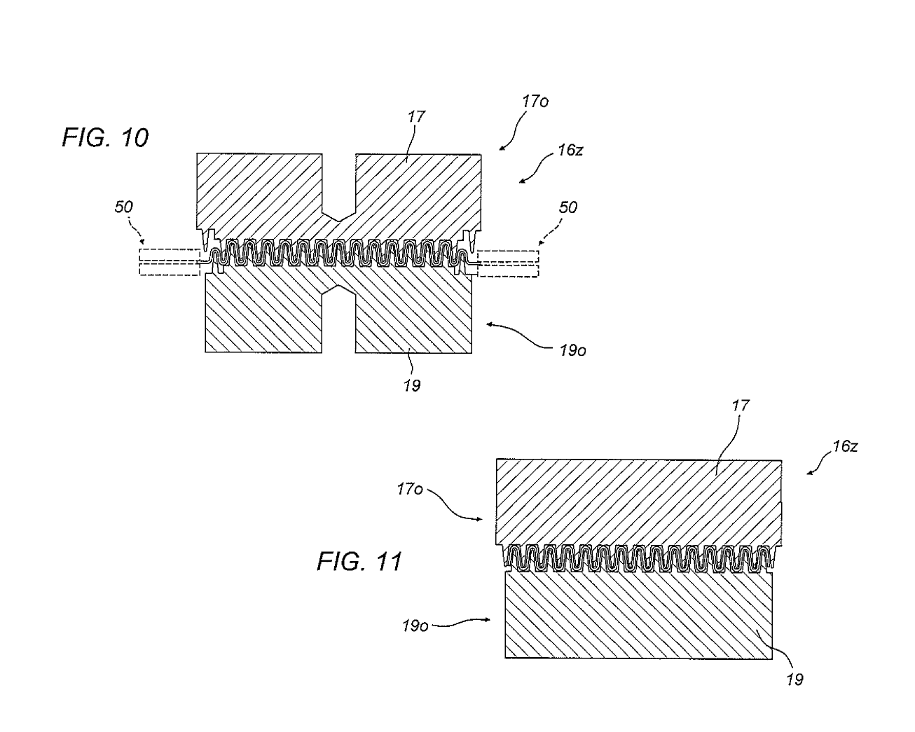

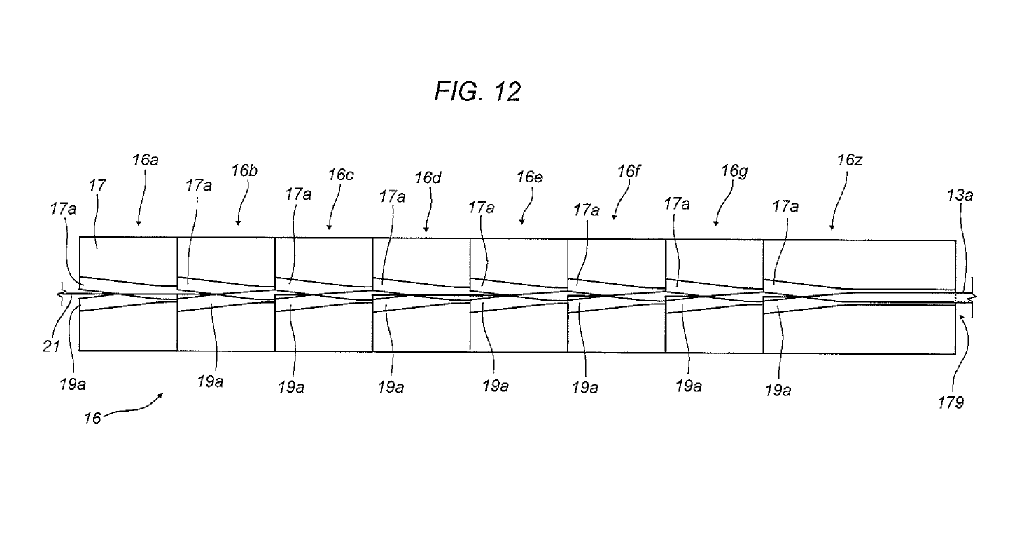

More specifically, as may be inferred from FIGS. 4 to 11, a single folding step entails making one pleat or pair of laps 41, 41 which have one fold line 41′ and which are, in particular, connected to parts of the basic layer 21 which are transversely on the outside of the respective pleat.

As illustrated in FIG. 1D, adjacent laps 41, 41, which are obtained by folding, make an angle with each other, in particular, an acute angle such as not to excessively stress the material during folding.

Folding the basic layer 21 thus entails performing a folding step, in particular a first or initial folding step, which entails making a single pleat portion or single pair of laps 41, 41, preferably at an intermediate zone of, and more specifically at a central zone of, the basic layer 21.

Further, folding the basic layer 21 also entails performing a final folding step and, preferably, one or more, (in particular, a series of) intermediate folding steps between the initial folding step, and the final folding step.

The first folding step, as mentioned, entails making a single pleat portion or single pair of laps 41, 41, at a central zone of, the basic layer 21, where the common fold line 41′ corresponds to a longitudinal centre line of the basic layer 21.

More specifically, the folding steps following the first folding step entail making a first and a second pair of laps simultaneously.

Indeed, more specifically, in an intermediate folding step, or in the final or last folding step, the respective folding step entails simultaneously making a first and a second pleat portion or first and second pairs of laps 41, 14.

The folding step, or more specifically, a step following the first folding step, entails simultaneously making a first and a second pleat portion or first and second pairs of laps, at an intermediate zone between the central zone where the laps have already been made, and the lateral edges 27, 29 of the basic layer 21.

The final folding step in turn entails simultaneously making a first and a second pleat portion or first and second pairs of laps, at opposite lateral end zones of the basic layer 21.

More specifically, a folding step following the first folding step, entails simultaneously making a first and a second pleat portion or first and second pairs of laps which are placed symmetrically about, or equidistant from, the initial fold line 41′ or the centre line of the basic layer 21, as illustrated for example in FIG. 6 and in the figures following it.

In practice, folding the basic layer 21, starting from a central zone of the layer 21, and proceeding towards the lateral edges 27, 29 of the basic layer 21 prevents creating transversal stress and strain which could split or tear the basic layer 21.

The portions, or laps, 41, 41 of the basic layer 21 have a height h′ which is equal or substantially equal to a corresponding height h′ of the intermediate mass 31.

Advantageously, according to the process, the intermediate mass 31 is obtained by moving closer together the portions, or laps, 41 of the basic layer 21.

More specifically, as illustrated in FIGS. 1A and 2A, the pleat portions which extend parallel to each other, in particular at the outfeed of the folding means 16, are made to converge or move closer together, proceeding downstream until reaching the infeed of compression means, in particular primary compression means, 30.

More specifically, the intermediate mass 31 is obtained by causing the laps 41, 41 of the basic layer 21 to move closer together and adhere to each other, preferably, as illustrated, starting from the pleated configuration shown in FIG. 1D.

In practice, the longitudinal faces 23 and 25 of the basic layer 21 possess a certain degree of adhesiveness. Thus, bringing the outside faces 41a, 41b of the laps 41 into contact with each other by suitable pressure, causes the selfsame laps 41 to adhere to each other to define an intermediate elongate mass or second intermediate mass 31b.

This intermediate mass, or second intermediate mass 31b, is thus obtained by compression of the portions, or laps, 41 against one another.

The compression of the portions, or laps, 41 against one another starting from the basic layer 21 is accomplished by a step of pre-compression, or primary compression, performed in the primary compression means 30.

Further, compression of the portions, or laps, 41 the basic layer 21 against one another is completed by a step of final compression, or compression proper, performed in compression means, in particular final compression means 40, in particular during a step of compression moulding the solid form 11.

The compression, and more specifically, both the primary compression or pre-compression, and the final compression or compression proper, of the portions, or laps, 41 of the basic layer 21 against one another is obtained by applying pressure along a direction which is transversal to the laps 41, or which is transversal to the direction of feed.

As illustrated, the solid form 11 is obtained from the elongate intermediate mass 31 by separation of the selfsame intermediate mass 31.

More specifically, the solid form 11 is obtained from the intermediate mass 31 by cutting, in particular by transversely cutting the intermediate mass 31.

As illustrated, the respective solid forms 11 are obtained from the intermediate mass 31 by separating or cutting them in succession from the elongate intermediate mass 31.

More in detail, the solid form 11 is obtained from the elongate intermediate mass 31 by compression moulding the intermediate mass 31 in respective compression moulding means 141, 42.

More specifically, the final compression of the intermediate mass 31 is performed during the step of separating the solid form 11, that is, during the step of compression moulding the solid form 11.

As illustrated, a first preferred embodiment of the apparatus 10 according to the invention comprises means, denoted in their entirety by the reference numeral 12, which are designed to make an intermediate mass of material 31 starting from a basic layer 21 of respective thickness t0 and respective width w0.

The apparatus 10 also comprises means 14 designed to obtain from the intermediate mass 31 a respective solid form 11 of material.

In practice, folding means 16 are provided which are designed to make a first intermediate mass 31a, which comprises a pleated layer, described above, obtained by deforming or folding the basic layer 21, and primary compression means 30 designed to make a second intermediate mass, or second part of the intermediate mass, 31b, which is obtained from the pleated layer and is in the form of a corresponding elongate body.

In a different embodiment, not illustrated but already mentioned above, the means which make the second intermediate mass 31b to define the elongate body may be omitted. In that case, the solid form 11 would be obtained directly from the pleated portion 31a thanks to compression moulding means 40. In that case, the pleat portions 41, 41 which extend parallel to each other, in particular at the outfeed of the folding means 16, would be made to converge, proceeding downstream until reaching the infeed of the compression moulding means 40.

As illustrated, the apparatus further comprises means 20 for feeding a respective basic layer 21. The feed means 20 may advantageously comprise a compensating buffer 22 for storing a reserve of material 21′ being fed to the means downstream for making the intermediate mass 31 and the solid form 11 of material.

As illustrated, the compensating buffer 22 is, more specifically, defined by respective rolls 22a, 22b, between which a reserve stretch 21′ of the basic layer 21 is allowed to sag.

In an alternative embodiment, not illustrated, the compensating buffer 22 might comprise a dandy roller system to keep the basic layer 21 tensioned.

Also provided are means for advancing the basic layer 21 and the elongate intermediate mass 31 and which, in particular, advance the basic layer 21 and the intermediate mass 31, with stepping, or advantageously continuous, motion along the feed direction.

According to the process, therefore, the basic layer 21 and the intermediate mass 31 are advanced in particular by continuous feed motion.

The means 12 for making the intermediate mass 31 comprise the folding means 16, which are adapted to define a plurality of portions, or laps, 41 of the material, in particular to define a first part 31a of the intermediate mass 31.

Further, the reference numeral 18 denotes means for aggregating the layers 41 of material to each other, in particular to define a second part 31b of the intermediate mass 31.

In practice, the steps of folding the basic layer 21, aggregating the intermediate mass 31 and forming the respective solid forms 11 are performed while the material advances, in particular while the material advances continuously.

More specifically, as may be inferred from FIG. 2A, the second intermediate mass 31b has a respective thickness or width t′, which is obtained from a plurality of layers or portions 41 of the basic layer 21 and is therefore greater than the thickness t0 of the layer of material 21.

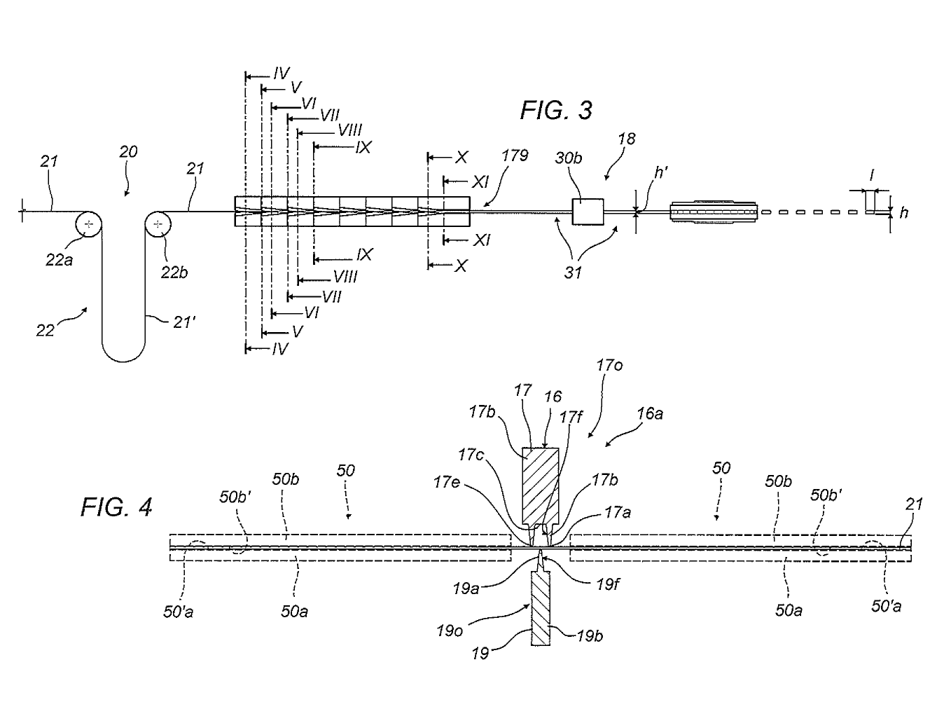

Further, as may be inferred from FIG. 3, the second intermediate mass 31b, has a height h′, which substantially corresponds to the height h′ of the laps 41 obtained from the basic layer 21.

The means 18 which aggregate the layers of material 41 are designed to cause the layers of material 41 to adhere to one another.

More specifically, with this apparatus the intermediate mass of material 31 which is obtained, in particular the second intermediate mass 31b which is obtained, is in the form of a continuous or elongate body.

Advantageously, the intermediate mass of material 31, or the second intermediate mass 31b, has a height h′ which is smaller than, in particular, slightly smaller than the thickness h of the solid form 11 to be made and/or has a thickness or width t′ which is greater, in particular slightly greater than, the width t of the solid form 11 to be made. All of this is done, preferably, in such a way as not to vary the density of the material during the step of compression moulding and shaping the solid form 11.

Further, as may be inferred from FIG. 2B, the length 1 of the solid form 11 is substantially the same as the length of a portion of the second intermediate mass 31b of the same weight.

The length “l” of the solid form 11 is suitably determined by a length, or rather, by a circumferential length of compression moulds 141, 42.

As illustrated, the apparatus 10 and, more specifically, the folding means 16 which are designed to define the layers of material 41 are fed with a single layer of material 21.

More specifically, the folding means 16 are designed to define layers which consist of portions, or strips, 41, in particular longitudinal portions, or strips, 41 of the basic layer 21.

More specifically, the folding means 16 are designed to define layers of material 41 which consist of portions, or strips, 41, 41 of the basic layer 21 which are adjacent to each other.

In practice, folding means 16 are provided which are designed to shape, or fold, the basic layer 21 in such a way as to define a corresponding intermediate mass 31.

More specifically, the folding means 16 extend one or more portions or laps, 41 perpendicularly to the plane L-T defined by the basic layer 21 in the plane condition.

More specifically, the folding means 16 are in the form of means designed to shape, or more specifically to fold, the basic layer 21.

In practice, the folding means 16 make in the selfsame basic layer 21 one or more pleat portions, each defined by a first and a second lap 41, 41 having a longitudinal fold line 41′ in common, each lap 41, 41 defining a respective layer of the intermediate mass 31.

As illustrated, the folding means 16 extend each lap 41 along a direction perpendicular, or substantially perpendicular, to the plane L-T defined by the basic layer 21 in the plane or unshaped condition, where the lap 41, in particular, protruding on the opposite side of the plane L-T.

As illustrated, in the embodiment being described, the laps 41 all have the same height h′ or substantially the same height h′.

More specifically, as mentioned above, inside the folding means 16, the laps 41 of a respective pleat portion make a respective angle with each other.

As illustrated, the means 16 for folding the basic layer 21 comprise means 17a, 19a for engaging the basic layer 21, these engagement means being designed to push respective pleat portions, or laps 41, 41 of the basic layer 21 perpendicularly to the plane L-T defined by the basic layer 21 in the unshaped condition.

More specifically, the means 16 for folding the basic layer 21 comprise respective engagement means 17a, protruding perpendicularly to a longitudinal face 23 of the basic layer, and respective second engagement means 19a, protruding perpendicularly to a second longitudinal face 25 of the basic layer 21.

As may be inferred from FIGS. 3 and from 4 to 11, the first and second engagement means 17a, 19a extend longitudinally along the feed direction, according to a respective profile which extends along the perpendicular direction P, between a position of minimum engagement of opposite longitudinal faces 23, 25 of the basic layer 21, when the respective pleat portion 41, 41 starts being folded, and a side-by-side position of transversal interference or maximum engagement, with respective laps 41, 41 of the basic layer 21 being interposed to define a corresponding pleated portion.

In practice, as illustrated, the folding means, or folder, 16 comprise a first and a second body 17, 19 located on opposite sides at the longitudinal faces 23, 25 of the basic layer 21. The first and second folding bodies 17, 19 have corresponding protrusions 17a, 19a, which extend longitudinally along the feed direction to define corresponding means for engaging and folding the pleated portions 41, 41 in the basic layer 21.

In practice, folding means 16 are provided which comprise corresponding protrusions 17a, 19a, extending longitudinally to define means for the engagement and shaping of, in particular for the pleating of, portions 41, 41 of the basic layer 21.

The first and second folding bodies 17, 19, opposed along the perpendicular direction P, have respective longitudinal plates 17b, 19b, from which extend corresponding protrusions defining the engagement means 17a, 19a.

The first and second bodies 17, 19 define, as a whole, respective folding sections of this the first preferred embodiment of the apparatus.

These shaping and folding means 16, in particular the first and second opposed bodies 17, 19, are in the form of fixed means and define between them a plurality of parallel, longitudinal channels or slots for folding the basic layer 21 passing through these channels.

The folding means 16 comprise corresponding protrusions 17a, 19a which extend along the perpendicular direction P to define means for the engagement and shaping of, in particular for the pleating of, portions 41, 41 of the basic layer 21.

The protrusions 17a, 17b have respective opposite faces 17f, 17f, 19f, 19f for engaging the basic layer 21 and which, in particular, converge along the direction of the basic layer 21 to be shaped.

In practice, the first and second opposed bodies 17, 19 have corresponding protrusions 17a, 19a, which intersect each other to define channels for the passage and folding of the transversal profile of the basic layer 21. The transversal cross section of the passage channels has a zigzag or broken line shape.

In practice, in the pleated configuration, the basic tape 21 has a zigzag, or broken line or wavy configuration.

The first and second opposed bodies 17, 19, or opposite folding end section elements, define a respective infeed upstream of the folding channels, and an outfeed 179, downstream, from which the layer of material 21, comes out in a folded or pleated condition.

As illustrated, the protrusions 17a, 19a have a wedge-shaped, or triangular, cross section, in particular with a rounded end which converges in the direction of opposed protrusions 19a, 17a, that is, towards the basic layer 21 to be shaped.

In practice, as illustrated, respective adjacent shaping protrusions 17a, 19a of the respective folding body 17, 19 are transversely spaced from each other and are connected, or joined, through a respective inner end surface 17c, 19c.

As illustrated, the inner end surface 17c, 19c extends longitudinally along the direction of feed according to a respective profile which extends between a position away from, and a position close to, the basic layer 21.

The shaping means 16, in particular the folding bodies 17, 19, have a respective initial section 16a, upstream, supporting the engagement means 17a, 19a which are designed to define a respective central pleat portion 41, 41 in the basic layer 21.

Further, the shaping means 16, in particular the folding bodies 17, 19, have a respective final section 16z, downstream, supporting the engagement means 17a, 19a for all the pleat portions made in the basic layer 21.

The shaping means 16, in particular the folding bodies 17, 19, also have one or more intermediate sections, in particular, a plurality of intermediate sections 16b, 16c, 16d, 16e, 16f, 16g, between the initial section 16a and the final section 16z, which support respective engagement means 17a, 19a designed to define corresponding pleat portions 41, 41 in the basic layer 21.

As illustrated, the folding means 16, in particular the respective folding bodies 17, 19, thus comprise a plurality of folding sections 16a, 16b, 16c, 16d, 16f, 16g, each folding section 16a, 16b, 16c, 16d, 16e, 16f, 16g being transversely narrower than the section immediately downstream of it.

More specifically, the initial folding section 16a makes a single pair of laps, or a single pleat portion 41, 41 and in particular, the initial folding section 16a makes a pair of laps, or pleat portion 41, 41 at a central zone of the basic layer 21.

Further, each intermediate folding section 16b, 16c, 16d, 16e, 16f, 16g simultaneously makes a first and a second pair of laps or a first and a second pleat portion 41, 41, which are, in particular, situated at an intermediate transversal zone between the central zone where the laps have already been made by the upstream sections, and a respective lateral end or zone of the basic layer 21. More specifically, the first and second pairs of laps and/or pleat portions 41, 41 made by the folding sections following the first one are arranged symmetrically about a longitudinal centre line of the basic layer 21.

The final folding section 16z simultaneously makes a first and second pair of laps or pleat portions 41, 41 at opposite lateral ends or zones of the basic layer 21.

For the purpose, the folding sections 16a, 16b, 16c, 16d, 16e, 16f, 16g, 16z have a suitable number of folding portions 17a, 19a.

Further, the folding sections 16b, 16c, 16d, 16e, 16f, 16g, 16z downstream of the first folding section 16a have respective intermediate folding protrusions 17a, 19a located between respective lateral protrusions. The intermediate folding protrusions 17a, 19a serve to maintain the pleated shape of the pleat portions made by the folding sections 16a, 16b, 16c, 16d, 16e, 16f, 16g immediately upstream.

In practice, each folding section 16a, 16b, 16c, 16d, 16e, 16f, 16g, 16z has, or is defined by, a first and a second folding member, or element, 17o, 19o, in particular a first, upper folding element 17o and a second, lower folding element 19o, which engage the basic layer 21 from opposite sides and which act in conjunction to make one or more corresponding shaped, or folded, portions, in particular, one or more pleated portions 41, 41.

More specifically, the number of folding sections is a function of the width w0 of the basic layer 21.

The laps 41 made in the basic layer 21 have a height h′ which is substantially the same as a height h′ of the intermediate mass 31, the latter being smaller than, in particular slightly smaller than the height h of the solid form 11 which is obtained from the intermediate mass 31 (see FIG. 2D).

The means 18 which are designed to aggregate the layers of material 41 advantageously comprise means 30 which are designed to move the folded laps 41, 41 of the basic layer 21 closer together to define the intermediate mass 31, in particular the second intermediate mass 31b.

The means 30 which are designed to move the folded laps 41, 41 of the basic layer 21 closer together are designed to cause the folded laps 41 to adhere to each other.

Means 30, 40 are also advantageously provided which are designed to compress the portions, or laps, 41, 41 of the basic layer 21 to define the intermediate mass 31, in particular the second intermediate mass 31b.

In practice, the means 18 which are designed to aggregate the layers of material comprise means 30 which are designed to compress the portions or laps 41 of the basic layer 21.

More specifically, there are primary compression means, or primary compressor, 30, in particular defining precompression means.

The primary compression means 30 comprise a pair of opposed primary compression rolls 30a, 30b, between which the layers or laps 41 of the basic layer 21 pass. The primary compression means 30 engage and compress the folded layers or laps 41 against each other to define the intermediate mass 31, or the second intermediate mass 31b.

As illustrated, the primary compression rolls 30a, 30b are rotatable about axes which are perpendicular to the plane L-T defined by the basic layer 21 in the plane condition, that is, axes which are parallel to the perpendicular axis P.

Also provided are final compression means, or presser, for the portions, or laps, 41 of the basic layer 21 to define a corresponding solid form 11.

Also provided are means 40 which are designed to separate the solid form 11 from the intermediate mass 31.

The separating means, or separator, 40 of the solid form 11 from the intermediate mass 31 are in the form of cutting means and are, in particular, in the form of means designed to perform a respective transversal cut in the intermediate mass 31.

Advantageously, the means 40 which are designed to obtain the solid form 11 of material from the intermediate mass 31 are in the form of means for compression moulding the intermediate mass 31.

The compression moulding means 40 also define final compression means for the intermediate mass 31.

The compression moulding means 40 comprise a respective mould having opposed, first and second parts 141, 42 acting in conjunction with each other.

The opposed parts 141, 42 of the mould are movable between a position where they are close together in order to enclose or mould a corresponding solid form 11 and a position where they are spaced apart in order to allow the solid form 11 to be expelled from the mould, that is to say, a spaced-apart position for releasing the solid form 11.

The opposed, first and second mould parts 141, 42 are carried on opposed means for advancing the selfsame mould parts.

More specifically, the opposed, first and second mould parts 141, 42 are carried on a pair of opposed wheels 40a, 40b. As illustrated, the intermediate mass 31 of material is interposed between the wheels 40a, 40b, that is, it is interposed between the opposed first and second parts 141, 42 of the mould.

In practice, the wheels 40a, 40b have a peripheral surface which is shaped to define the opposed first and second moulding parts 141, 42.

More specifically, on the respective wheels 40a, 40b, the opposed, first and second mould parts 141, 42 are contiguous.

Further, as illustrated in FIGS. 2B and 13, each mould part 141, 42 comprises a plurality of contiguous half moulds. Each half mould comprises a transversal leading end wall 43a, in common with the half mould before it, a trailing end wall 43b, in common with the half mould after it, and a pair of opposite longitudinal walls 43c and 43d.

Further, the respective half mould has an inner wall 43e, which is recessed relative to an outer profile of the half mould and extends between the longitudinal walls 43c, 43d and between the leading end wall 43a and the trailing end wall 43b relative to which it is suitably radiused. As illustrated, according to this preferred embodiment, the inner wall 43e has a circular profile.

The first and second mould parts 141, 42 define compressing means for the layers, or laps, 41 of material.

The opposed first and second mould parts 141, 42 thus define compressing means for the layers, or laps, 41, 41 of material.

Further, the wheels 40a, 40b which carry the opposed first and second mould parts 141, 42 are rotatable about axes which are perpendicular to the plane L-T defined by the basic layer 21 in the plane or unshaped condition, that is, axes which are parallel to the perpendicular axis P.

It is understood, however, that the mould, or the half moulds, might have any suitable shape desired.

By way of an example, there might be moulding means movable in a straight line transversely to the intermediate mass 31, for example along the transversal direction T. These moulding means might also be movable in a straight line along the feed direction in order to work by following the intermediate mass 31.

Also provided are means for pulling the intermediate mass 31 and/or the basic layer 21.

In the embodiment illustrated, the primary compression means 30 and the moulding means 40 act as pulling means.

As illustrated, the pulling means are downstream of the means 16 for folding the basic layer 21 and cause the basic layer 21 to advance between the fixed folding means 16.

Although this embodiment is especially preferred, in another embodiment not illustrated, other suitable pulling means might be provided.

This process and apparatus, make it possible to obtain a solid form of material starting from a basic layer 21 which can be made using suitable, simpler methods, for example, working on the liquid phase which allows the ingredients or raw materials to be distributed uniformly more easily in the selfsame basic layer 21, thus allowing a solid form 11 with an optimum composition to be made.

Further, this embodiment of the apparatus is advantageously simplified in terms of structure and has a limited construction cost.

With this apparatus, the solid form 11 is obtained by compression moulding from a respective portion of the intermediate mass 31, preferably without modifying or without substantially modifying the density thereof.

In effect, as may be inferred from FIGS. 2C and 2D, which show respective side and transversal views of the solid form compared to a corresponding portion of the intermediate mass of the same weight (shown in dashed lines) from which it is obtained, a solid form 11 is provided which has a width t which is smaller than the width t′ of the portion of intermediate mass 31 it is obtained from, compensated by a height h of the solid form 11 which is correspondingly greater than a height h′ of the intermediate mass 31 it is obtained from.

In short, in the case of solid forms with density equal or substantially equal to the intermediate mass they are obtained from, the solid form 11 and the corresponding intermediate mass 31 have transversal cross sections which are equal in area but different in dimensions on account of the compression moulding step.

It is understood that the solid form made with this process and apparatus is preferably obtained from a basic film or layer which is in a sufficiently dry or solidified state when it feeds into the apparatus and whose mechanical properties are such as to prevent it from being torn and split during subsequent operating steps, especially during the steps of forming the compensating buffer and/or shaping or folding.

It is also understood that the solid form made with this process and apparatus is preferably obtained from a basic film or layer whose tensile strength is such that it does not tear or split and, moreover, whose surface must have a low friction coefficient so as to minimize resistance to sliding, especially inside the folding means.

It is also understood that the solid form made with this process and apparatus is preferably obtained from a basic film or layer whose plastic properties are such that it keeps the folds made in the intermediate mass and/or whose adhesive properties are such that the longitudinal faces of the basic layer adhere to each other when placed in contact.

As may be inferred from FIG. 4 and following, the apparatus comprises, at the shaping means 16, means 50 which are adapted to define a guide plane in which the basic layer 21 lies.

More specifically, as illustrated, the means 50 which are adapted to define a guide plane, are located laterally of the folding means 16 and comprise means 50a, 50b for engaging the portion of the basic layer 21 protruding laterally from the folding means 16.

More specifically, the means 50 which are adapted to define a guide plane, comprise a lower element 50a defining a longitudinal supporting surface 50′a for the basic layer 21.

More specifically, the means 50 which are adapted to define a guide plane, comprise an upper element 50b defining a longitudinal retaining surface 50′b for the selfsame basic layer 21.

According to another viewpoint, the means which are adapted to define a guide plane, comprise opposed first and second elements 50a, 50b defining facing longitudinal surfaces 50′a, 50′b for engaging the layer of material which slides between the engagement surfaces 50′a, 50′b themselves.

This ensures that deformation or folding of the layers of material occurs at the folding or shaping means.

In another embodiment, not illustrated in the accompanying drawings, at least the upper retaining element 50b of the guide plane means might, however, be omitted.

Below is a brief description of how the apparatus of the invention operates. The basic layer, or film, 21, which is in particular, in the form of a continuous band or tape and whose thickness is negligible relative to its width, is fed along a feed direction into folding means, or folder, 16 from which it comes out in folded configuration defined by a plurality of pleated portions, or laps, folded perpendicularly to a plane L-T defined by the basic layer 21 in the plane condition, that is, to the feed direction L.

Next, the tape of material 21 in the pleated or folded lap condition, enters corresponding presser means, or aggregator or presser, 30, which transversely press the folded laps of the basic layer 21 to define a compact configuration or elongate body 31b, which is fed to compression moulding means, or former, 40, from which the solid forms, or tablets, 11 of material are fed out.

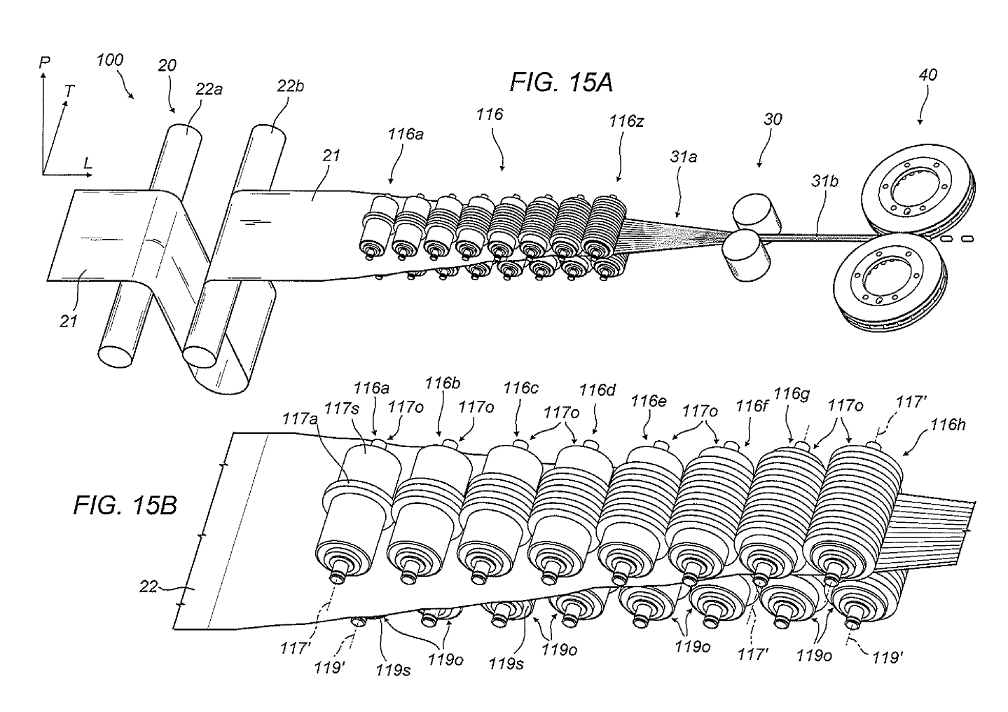

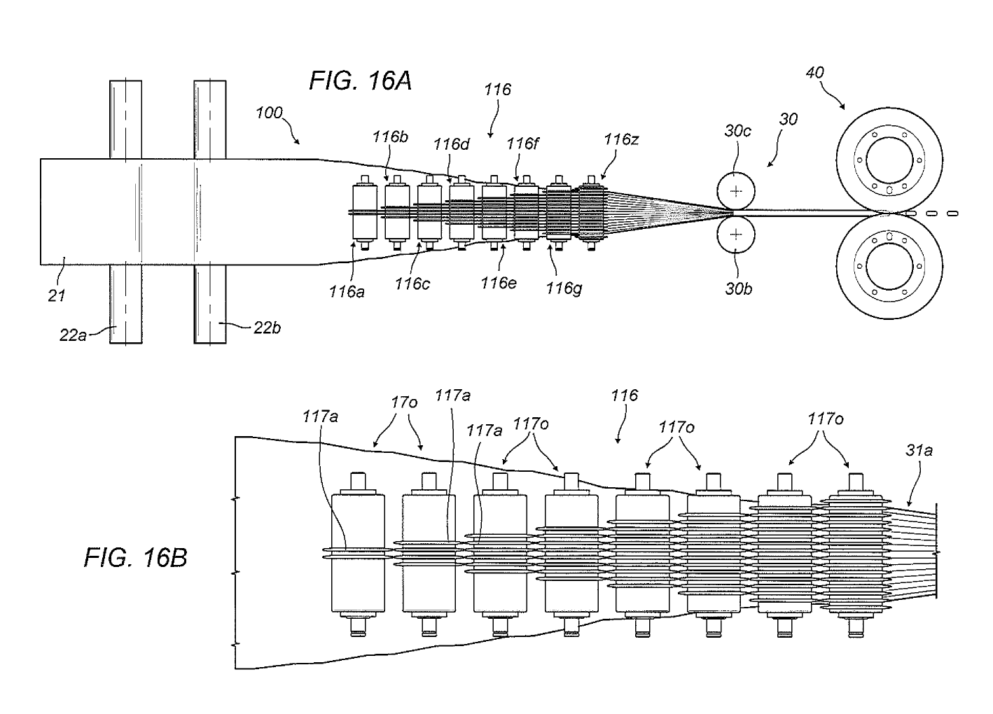

FIGS. 15A to 18E show a second preferred embodiment 100 of the apparatus which implements the advantageous process for obtaining the solid form 11 described above.

The second preferred embodiment 100 of the apparatus comprises components which are identical, or can be fully likened, to those of the first preferred embodiment, which are denoted by the same reference numerals as those and which will not be commented upon again in detail in order to avoid making this description too lengthy.

More specifically, the second preferred embodiment 100 comprises folding means 116 by which, in this case, too, the basic layer 21 is folded and which are suitable for making a corresponding intermediate mass 31 of material. The folding means 116 are provided on opposite sides at the longitudinal faces 23, 25 of the basic layer base 21 and have respective protrusions 117a, 119a, which extend perpendicularly to the basic layer 21 to define engagement and shaping means designed to fold and, more specifically, to pleat respective portions 41, 41 of the basic layer 21.

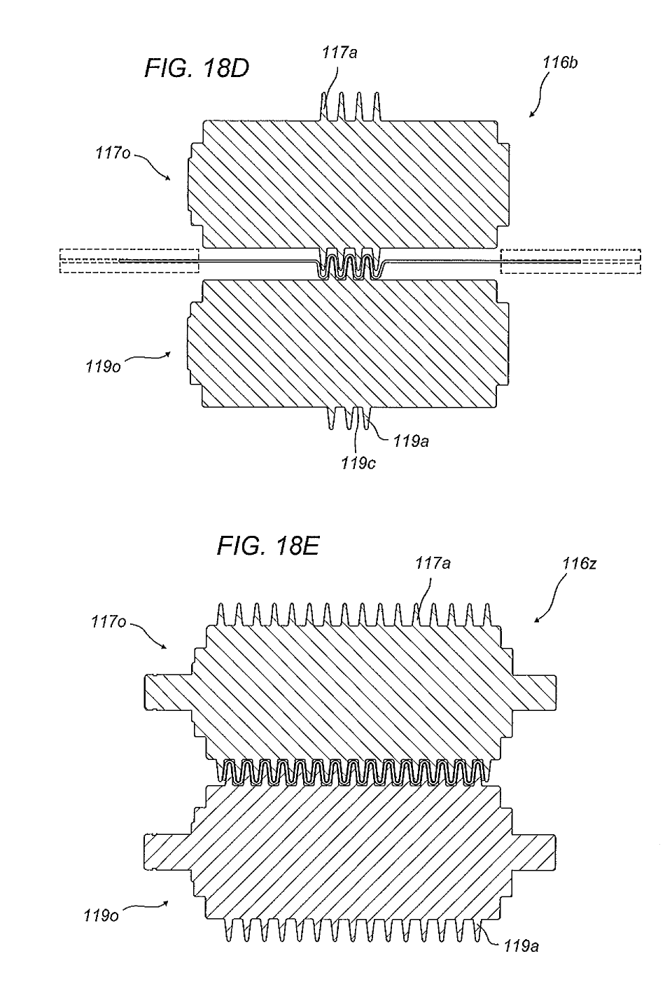

More specifically, as may be inferred from FIGS. 18C to 18E, in the second preferred embodiment, too, the protrusions 117a, 119a have a wedge-shaped, or triangular, cross section which converges towards the basic layer 21 to be folded.

More specifically, as may be inferred from FIG. 18C, in the second preferred embodiment, too, the protrusions 117a, 119a have opposite faces 117f, 117f, 119f, 119f for engaging the material, these faces converging in the direction of the basic layer 21.

More specifically, as may be inferred from the drawings, in the second preferred embodiment, too, the folding means 116 comprise a respective initial section 116a, upstream, which has respective engagement means 117a, 119a which are designed to define a respective pleat portion, in particular centrally of the basic layer 21.

Further, in the second preferred embodiment, too, the folding means 116 comprise a respective final folding section 116z, downstream, which has respective engagement means 117a, 119a which are designed to define respective pleat portions in the basic layer 21.

Further, in the second preferred embodiment, too, the folding means 116 comprise one or more intermediate folding sections 116b, 116c, 116d, 116e, 116f, 116g, between the initial section 116a and the final section 116z, these intermediate folding sections having engagement means 117a, 119a defining respective protrusions for engaging the basic layer 21.

It shall be understood, however, that in both the second preferred embodiment and the first preferred embodiment described above, the total number of folding or shaping sections may vary, in particular as a function of the width w0 of the basic layer 21, and also as a function of the size of the solid form 11 to be made.

In the second preferred embodiment, each shaping section 116a, 116b, 116c, 116d, 116e, 116f, 116g, 116z of the folding means 116 comprises a first and a second shaping member, or element, 117o, 119o, in particular a first, upper shaping element 117o and a second, lower shaping element 119o, which engage the basic layer 21 on its opposite longitudinal faces 23, 25 and which act in conjunction to make one or more corresponding shaped portions, in particular, one or more pleated portions 41.

Further, in the second preferred embodiment, too, adjacent protrusions 117a, 119a are transversely spaced from each other and are connected through respective inner end surfaces 117c, 119c joined to opposed faces 117f, 117f of transversely adjacent protrusions 117a, 119a.

Advantageously, the folding means 116 are in the form of movable means and, more specifically, in the form of means which are movable along with the basic layer 21 in the direction of feed of the basic layer 21.

In practice, the folding means 116 have a respective engagement protrusion which moves along with the material to be folded between an upstream position where it engages the material and a downstream position where it disengages the material, in particular after the latter has been conveniently folded, and then returns to the upstream position where it engages the material.

More specifically, the folding means 116 are movable rotatably, or rotate, preferably about a respective axis parallel to the plane L-T defined by the basic layer 21 in the plane condition, that is to say, parallel to the transversal direction T.

More specifically, each shaping section 116a, 116b, 116c, 116d, 116e, 116f, 116g, 116z has a first and a second shaping member, or element, 117o, 119o, of which at least one is in the form of a movable member, in particular movable rotatably.

More in detail, each shaping section comprises opposed first and second rotatable shaping members 117o, 119o between which the basic layer 21 extends and is folded.

In practice, the opposed rotatable shaping members 117o, 119o of one forming section define at least one respective folding section of the basic layer 21. The shaping channel is defined by opposed, intersecting circumferential lips 117a, 119a protruding from the opposed members 117o, 119o.

As illustrated, the rotatable member 117o, 119o of each forming section has a body which is rotatable about a respective axis 117′, 119′, and which has a respective peripheral surface 117s and 119s—in particular cylindrical—from which extend one or more circumferentially extending radial lips 117a, 119a for engaging the material to be shaped, each radial lip 117a, 119a defining a respective protruding portion for engaging the basic layer 21.

As illustrated each shaping section 116a, 116b, 116c, 116d, 116e, 116f, 116g, 116z has respective opposed first and second rotatable shaping members 117o, 119o whose respective axes of rotation 117′ 119′ are aligned with each other along the perpendicular direction P.

Further, the rotatable members, or rolls, 117o, 119o which carry the radial engagement lips 117a, 119a, and/or the radial engagement lips themselves are the same in diameter or width.

As illustrated, the folding means 116 comprise, in particular for each longitudinal face 23, 25 of the basic layer 21, a plurality of shaping rolls 117o and 119o which are aligned with each other in a row along the feed direction L.

In another embodiment, not illustrated in the drawings, the row of shaping rolls 117o and 119o, instead of being aligned in a straight line, might be aligned according to a circular line.

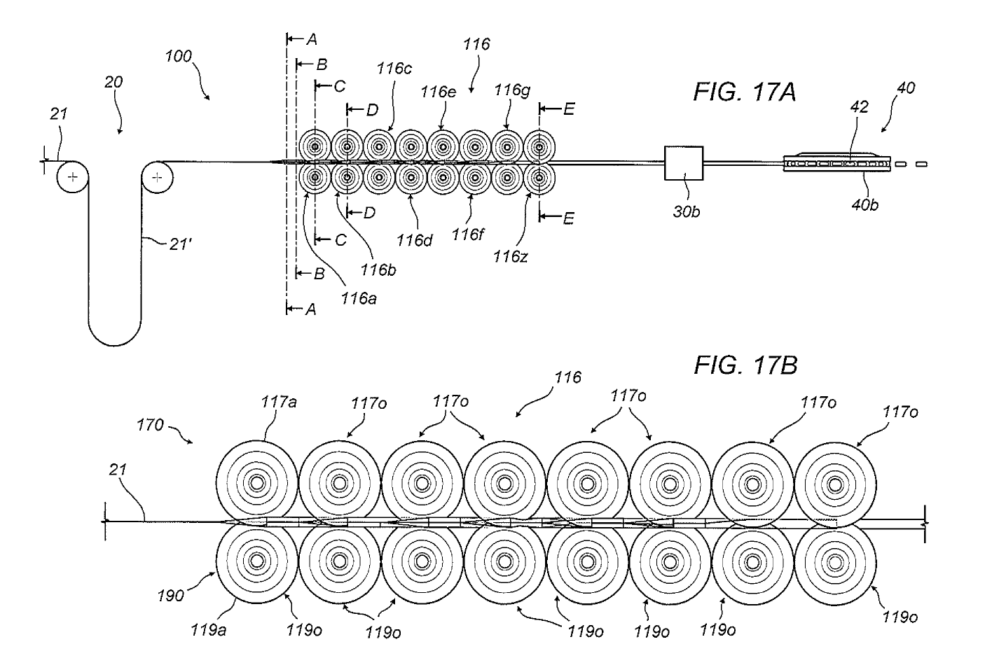

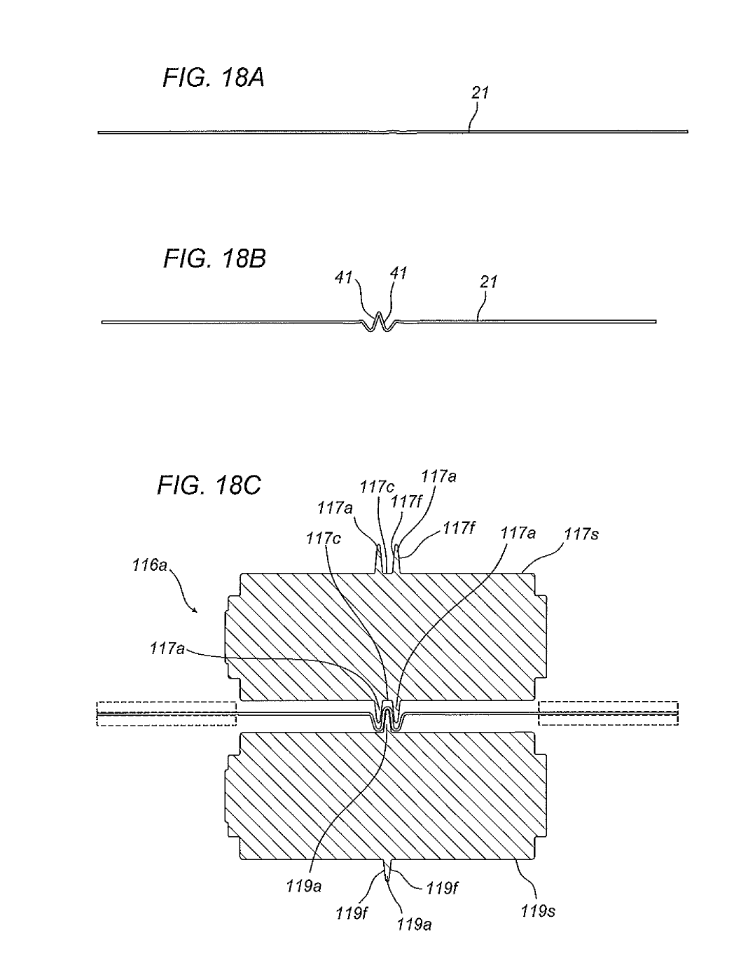

As may be inferred in particular from FIG. 17B, the engagement lips 117a, or 119a, of members 117o, or 119a, which are adjacent to each other along the feed direction L are aligned with each other in height.

In the second preferred embodiment, too, the initial folding section 116a makes a pair of laps, or a pleat portion, in particular, a pair of laps, or a pleat portion 41, at a central zone of the basic layer 21, whilst each intermediate folding section 116b, 116c, 116d, 116e, 116f, 116g simultaneously makes a first and a second pair of laps, or a first and second pleat portion 41, situated at an intermediate transversal zone between the central zone, where the laps 41, 41 have already been made, and lateral zones of the basic layer 21.

Lastly, the final section 116z simultaneously makes a first and second pair of laps or pleat portions 41 at opposite lateral end zones of the basic layer 21.

The second preferred embodiment advantageously makes it possible to reduce friction between the folding means 116 and the basic layer 21, thus speeding up work, saving energy and reducing stress in the basic layer 21.

The second preferred embodiment is therefore particularly suitable for processing a basic layer of material which has relatively little resistance to wear and mechanical stress.

As may be inferred from FIGS. 18A and 18B, a fold or partly pleated portion 41, 41 is made in the basic layer 21, upstream of the folding means 116.

In both of the above described embodiments of the apparatus, the basic layer 21 and the intermediate mass 31 are fed with continuous or mainly continuous motion under the pulling action exerted by the pulling means 30 and 40, which are in the form of respective pulling rolls or wheels, defining respective aggregators and formers.

It is also possible to feed the basic layer 21 and the intermediate mass 31 with discontinuous, or stepping, feed motion.

The invention thus provides an advantageous solid form of material, where the material is, in particular, a therapeutic or pharmaceutical product, a dietary supplement, and/or a food or edible product, the solid form comprising a plurality of layers 41 aggregated with each other, in particular, adherent to each other, and preferably obtained by folding a basic layer of the material.

Advantageously, a solid form 11 is provided which is obtained by compression moulding an intermediate mass 31 comprising a plurality of layers 41 aggregated with each other and obtained by pleating a basic layer 21 of the material.

The embodiments described above may be modified without departing from the scope of the invention.

By way of an example, in an embodiment not illustrated, the folding means 16, and more specifically, the engagement means 17a, 19a may be shaped in such a way that their protrusions along the perpendicular direction P vary in size along the transversal direction T in order to obtain an intermediate mass 31 with a differently shaped transversal cross section, for example circular or ovoid.

In other words, the engagement means 17a, 19a used may be shaped in such a way as to make laps 41 of different heights h′ in order to obtain an intermediate mass 31 with a transversal cross section of desired shape, for example, circular, ovoid, rhomboid, hexagonal, rectangular, etc. In this embodiment, the primary compression means (if provided), the final compression means, the pulling means and the separating means are suitably shaped to match the intermediate mass. More specifically, in the embodiment comprising compression moulding means, for example of the type comprising a mould with a first and a second part of the type illustrated in FIG. 13, the half moulds are suitably shaped to match the intermediate mass 31 to make the solid form 11 in a desired shape.

The invention can also be implemented on a basic layer comprising two or more superposed layers adherent to one another.

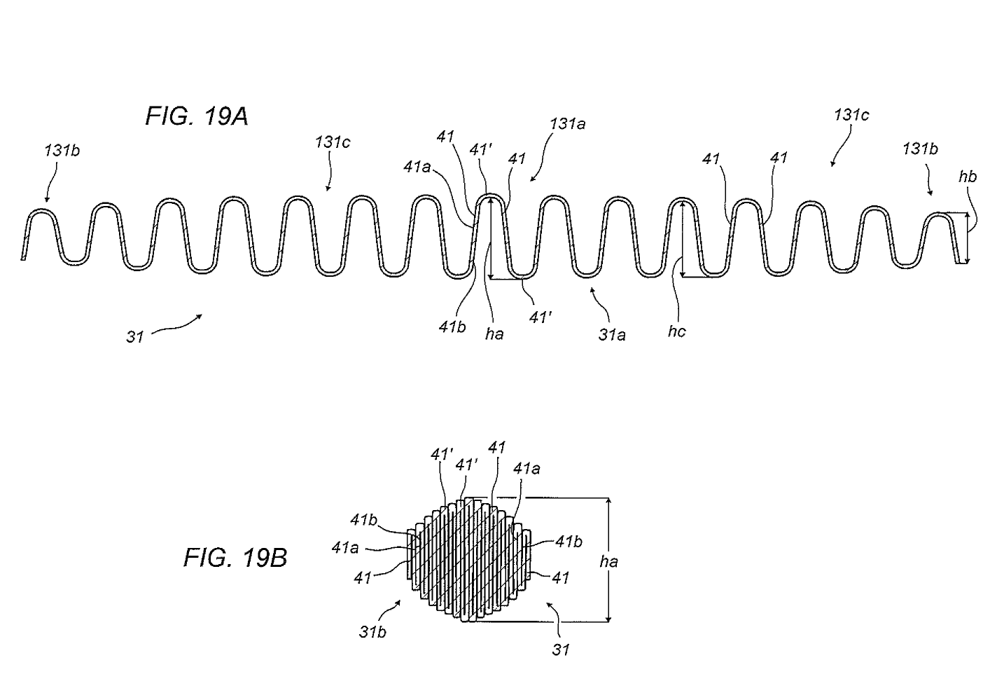

As may be inferred from FIGS. 19A and 19B, the intermediate mass 31, and hence the solid form 11 obtained therefrom may be given a desired shape.

To make such an intermediate mass 31, according to a further embodiment of the apparatus, which is not, however, illustrated in detail in the accompanying drawings, the folding means 116 are advantageously adapted to provide the intermediate mass 31 with respective laps or layers 41 which differ in height from each other.

More specifically, the folding means 116 are adapted to make an intermediate mass 31 whose respective laps, or layers, 41 have a maximum height “ha” in the central zone 131a of the intermediate mass 31, a minimum height “hb”, in the lateral zones 131b, 131b of the intermediate mass 31, and a respective height “hc”, between the maximum height “ha” and the minimum height “hb”, at the laps in the intermediate zones 131c, 131c of the intermediate mass between the central zone 131a and the lateral zones 131b, 131b.

More specifically, and preferably, as illustrated, the height “hc” of the intermediate laps decreases progressively starting from the central zone 131a towards the lateral zones 131b, 131b of the intermediate mass.

In other words, folding means 116 are advantageously provided which are adapted to provide, between adjacent fold lines 41′, a variable perpendicular distance, and more specifically, a maximum distance in the central zone 131a of the intermediate mass 31, a minimum distance at the lateral zones 131b, 131b of the intermediate mass 31 and a distance between the maximum distance and the minimum distance at the intermediate zones 131c, 131c located between the central zone 131a and the respective lateral zones 131b, 131b of the intermediate mass.

The perpendicular distance between adjacent fold lines progressively decreases starting from the central zone 131a of the intermediate mass towards each of the lateral zones 131b, 131b of the intermediate mass 31 itself.

Means might also be provided for adjusting the perpendicular distance between opposed members, or elements, 117o, 119o of each folding section, such adjustment means not being illustrated in detail in the accompanying drawings.

More specifically, means are provided for adjusting the perpendicular distance between opposed rotatable members, or elements 117o, 119o, of the respective section of the means 116 for folding or shaping the basic layer 21.

In particular, the upper shaping elements, or members 117o are moved, while the lower shaping elements, or members 119o are kept perpendicularly fixed.

Advantageously, each pair of opposed shaping elements, or members, 117o, 119o of the respective section is perpendicularly adjustable independently of the shaping elements, or members, 117o, 119o of the other sections.

Preferably, however, the pairs of opposed shaping elements, or members, 117o, 119o are perpendicularly adjustable, or positionable, in such a way that the first pair of opposed shaping elements, or members, 116a is spaced at a minimum distance from each other and the last pair of opposed shaping elements, or members, 116z is spaced at a maximum distance from each other, each of the intermediate pairs of opposed shaping elements, or members 116b, 116c, 116d, 116e, 116f, 116g being spaced at a respective distance which is between the minimum distance and the maximum distance and which preferably increases progressively starting from the minimum distance of the first pair of opposed shaping elements, or members 116a towards the last pair opposed shaping elements, or members 116z.

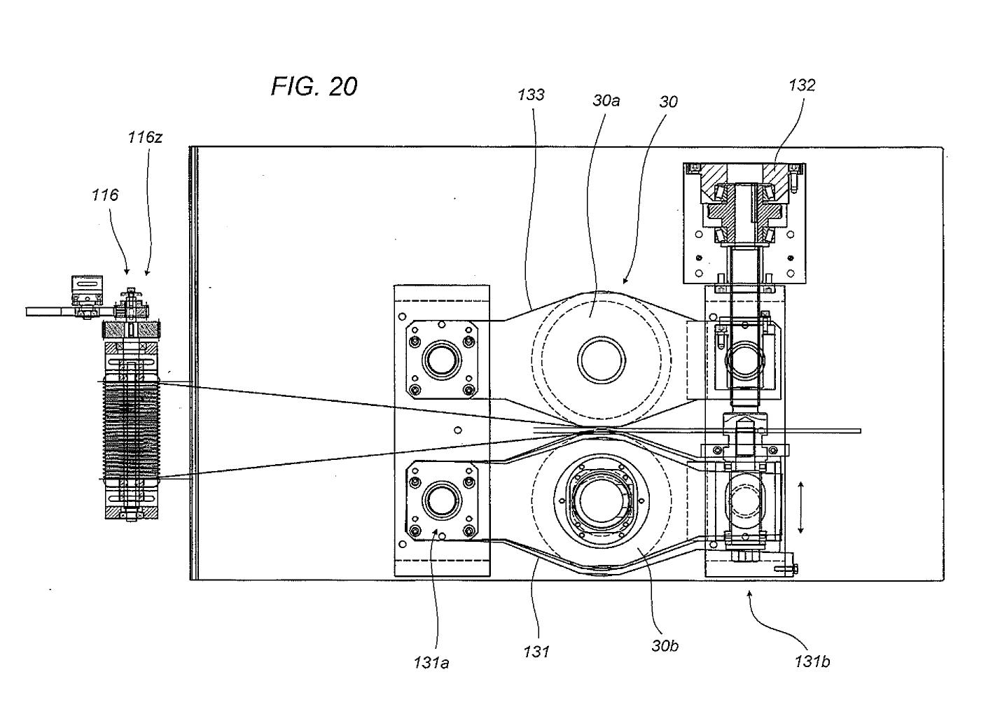

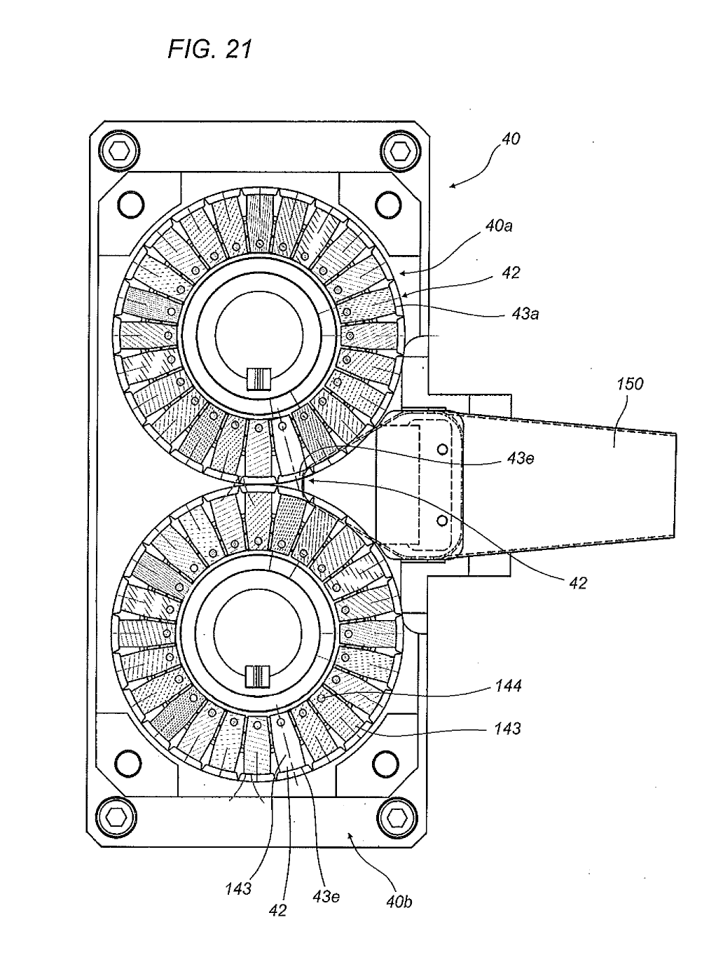

FIG. 20 illustrates a further preferred embodiment of the apparatus according to the invention, which is very much the same as the second preferred embodiment described above and whose components in common with the second preferred embodiment are denoted by the same reference numerals as those used for the components of the second preferred embodiment and possibly also of the first preferred embodiment. To avoid making this description too lengthy, the components which are in common with the preferred embodiments described above are not commented upon again in detail.

The further preferred embodiment differs from the other embodiments described above in that it comprises means for adjusting the compression applied to the intermediate mass 31 by the means 30 for the primary compression or aggregation or precompression of the intermediate mass 31.

More specifically, the compression adjustment means are designed to vary the spacing between the rolls 30a and 30b defining the means 30 for the aggregation or precompression of the intermediate mass 31.

Advantageously, as may be inferred from FIG. 20, the means 30 for the aggregation or precompression or primary compression of the intermediate mass 31 comprise a compression roll 30b, which is movable in particular along the transversal direction “T”, relative to the other compression roll 30a opposite it.

More specifically, one aggregation or precompression roll 30b is supported by a respective arm 131 which is pivoted at one end 131a and has a free opposite end 131b which is movable, in particular movable along the transversal direction “T”, since it is slidable on corresponding guide means, and which is operatively connected, through corresponding motion transmission means, to respective drive means, or motor, denoted by the reference numeral 132 in FIG. 20.

The arm 131 extends along the longitudinal direction “L” and supports the aggregation or precompression roll 30b rotatable about a respective perpendicular axis, that is, an axis parallel to the perpendicular direction “P”.

As may be inferred from FIG. 20, also the fixed roll, and more specifically the transversely fixed roll 30a is supported rotatably about an axis perpendicular to a longitudinally elongate arm 133, which is generally parallel to the arm 131 that supports the other compression roll 30b.

More specifically, the roll 30b is conveniently motor-driven in rotation, whereas the roll 30a rotates idly, that is, it is in the form of a non-drive roll.

It is understood that the transversely fixed roll 30a might be motor-driven and the movable roll 30b left idle, or that both the transversely fixed roll 30a and the movable roll 30b might be motor-driven.

Also advantageously provided are means which detect the state of compression of the intermediate mass 31 at the aggregation or precompression means and which are, in particular, defined by a respective load cell mounted on the transversely fixed roll 30a.

These detecting means emit a signal as a function of the force exerted by the movable roll 30b on the transversely fixed roll 30a. The signal is used by corresponding control means, in particular by the control means of the apparatus, to control the spacing between the aggregation or precompression rolls 30a and 30b, that is, to suitably adjust the compression or state of compression applied by the precompression or primary compression means 30 on the intermediate mass 31.

According to another advantageous aspect, means are provided for regulating the temperature of the intermediate mass 31.

More specifically, there are means for detecting the temperature of the intermediate mass 31—which are connected to corresponding control means—and means for thermally conditioning, in particular for heating and/or cooling, the intermediate mass 31, driven by the control means, which are, more particularly, the control means of the apparatus.

Preferably, the means for regulating the temperature of the intermediate mass 31 comprise conditioning means which are in the form of means for heating the intermediate mass 31. For example, to facilitate the aggregation of the layers, or laps, 41 of the intermediate mass, the latter might be heated to a temperature around 50° C., or in any case to a temperature higher than ambient temperature.

Advantageously, the means for regulating the temperature of the intermediate mass 31 are located at the aggregation or precompression or primary compression means 30.

More specifically, the means for regulating the temperature of the intermediate mass 31 are designed to facilitate compaction and aggregation of the layers, or laps, 41 of the intermediate mass 31.

More specifically, means are provided for detecting the temperature of the respective roll 30a, or 30b, of the aggregation or precompression or primary compression means 30.

Further, the means for regulating the temperature of the intermediate mass 31 are designed to thermally condition, or heat, a respective roll 30a and/or 30b of the means 30 for the aggregation or precompression of the intermediate mass 31.

More specifically, the roll 30a and/or 30b is heated through a respective electrical resistance element, conveniently located at the periphery of the respective roll and designed to come into contact with the mass of material to be aggregated. The electrical resistance element is conveniently driven by the electronic processing or control unit according to feedback from the temperature detection means, and more specifically, from the means for the detection of the temperature of the selfsame compression roll. It is understood, however, that the heated roll might also be provided with heating means of a different kind.

For example, a circumferential conduit might advantageously be provided inside the body of the roll 30a and/or 30b, the conduit being designed to have a heating fluid, for example an oil, flowing through it, or the body of the roll might be heated by electromagnetic induction by providing the interior of the structure, or body of the aggregation roll with electrical windings which can be energized by a magnetic field.

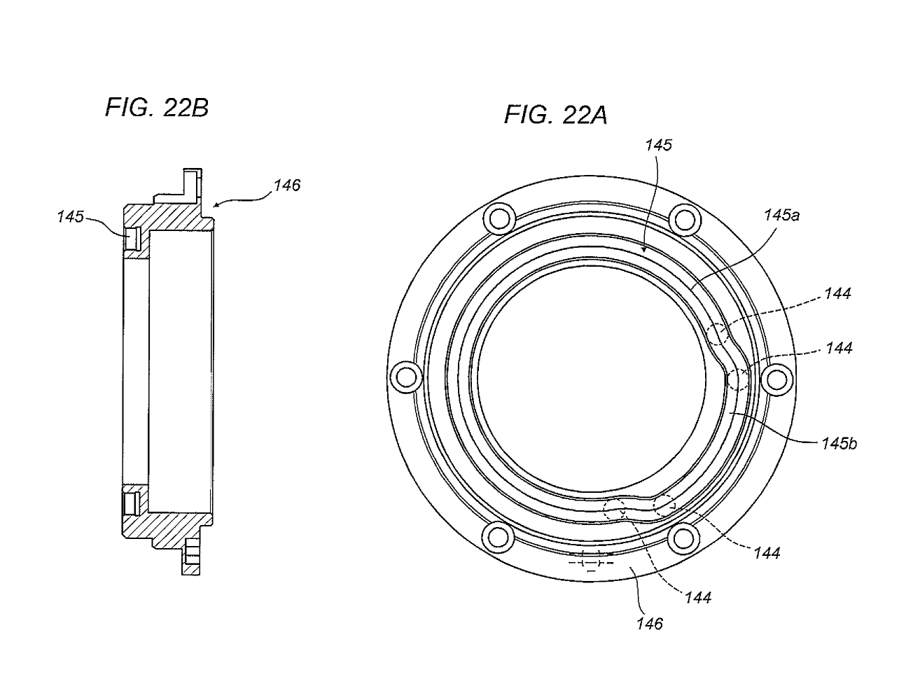

As may be inferred in particular from FIGS. 21, 22A and 22B, means are advantageously provided for expelling the solid form 11 from the forming means 40, in particular from the respective forming mould 40.

Advantageously, the means for expelling the solid form 11 comprise pushing means by which the solid form 11 is expelled from the respective cavity 42 of the mould or half mould 40 where it is formed.

Advantageously, the means for expelling the solid form 11 comprise the inner end wall 43e of the respective cavity 42 of the mould or half mould, on the respective moulding wheel 40a, 40b, the inner end wall 43e being conveniently movable between a retracted moulding position, illustrated in FIG. 21, and an extended position for releasing or expelling the solid form, located just downstream of the zone where the solid form 11 is moulded, and which is not illustrated in detail in FIG. 21.

More specifically, the means for expelling the solid form 11 comprise the inner end wall 43e which is part of the cavity of the respective mould or half mould 40a, 40b and which is movable between a retracted moulding position and a position for releasing or expelling the solid form 11.

It is understood, however, that by movable inner end wall 43e of the respective mould cavity 42 is meant either the entire inner end wall 43e, as described above, or a movable part of the inner end wall 43e, that is, any other cavity 42 part or wall which is movable in such a way as to facilitate expulsion of the solid form 11 from the cavity 42.

As may be inferred from FIG. 21, the inner end wall 43e of the mould or half mould cavity 42 of the moulding means 40 is connected, in particular is integral with, a radial block, or slider, 143 that mounts a respective roller, or similar element, 144, which is slidable on, or in, a corresponding cam 145 which is designed to control the movement, in particular the radial movement, of the inner end wall 43e between the retracted moulding position and the extended position for releasing or expelling the solid form 11. The cam is denoted by the reference numeral 145 and is illustrated in FIGS. 22A and 22B.

In FIG. 21 all the cavities of the moulding wheels are shown with the respective inner end walls in the retracted condition and with the drive sliders not yet interacting with the corresponding drive cams.

As illustrated, the drive cam 145 is made within a corresponding fixed profile 146 which is conveniently positioned on a rotatable moulding wheel 40a, 40b.

As illustrated, the cam 145 has a long main circumferential stretch 145a, largely circular in shape, by which the inner end wall 43e of the cavity 42 is placed in the retracted moulding position, and a short circumferential stretch 145b, which is radially outside the main stretch 145a and by which the inner end wall 43e of the cavity 42 is placed in the position for releasing or expelling the solid form 11 from the mould, or half mould, of the moulding means 40.

Advantageously, means are also provided for conveying the solid form 11 feeding out of the apparatus, these outfeed conveying means comprising a respective chute 150, downstream of the moulding means 40, in particular immediately downstream of the moulding means 40, the conveying means receiving the solid form 11 by gravity and running longitudinally along the apparatus towards a corresponding zone or container which receives the solid forms 11. The chute 150 is directed downwards at a suitable angle.

Advantageously, means are provided, in particular at the moulding means 40, for emitting a jet a fluid, in particular a jet of fluid which is directed from the top down and which is in the form of air under high pressure, this jet of fluid constituting corresponding, or additional, means for expelling the solid form 11 from the moulding means 40.

The jet of fluid under pressure is designed to direct the solid form 11 towards the means 150 for conveying the solid form 11 towards the outfeed of the apparatus, that is to say, it is designed to convey the solid form 11 directly to the outfeed chute 150.

According to another embodiment, not illustrated, the use of means for supporting the intermediate mass upstream and/or downstream of the aggregation or precompression means 30 is also imaginable, in particular between the selfsame means 30 and the folding means 16 and/or between the means 30 and the moulding means 40. Preferably, the supporting means are in the form of a surface on which to rest the intermediate mass upstream and/or downstream of the aggregation or precompression means 30.

The invention described above is susceptible of industrial application. It would be obvious to one skilled in the art that several changes and/or modifications can be made to the invention without departing from the spirit and scope of the invention, described in depth above. Also, further embodiments of the invention comprising one or more of the features described herein can be easily imagined. It will also be understood that all the details of the invention may be substituted by technically equivalent elements.

1. A process for obtaining a solid form (11) of material, said material being a therapeutic or pharmaceutical product, a dietary supplement, and/or a food or edible product, wherein it comprises, starting from at least one basic layer (21) of the material in the form of a sheet or band, a step of folding the basic layer (21) in order to make a plurality of portions or laps (41) defining an intermediate mass (31) of material from which the solid form (11) is obtained by separation.

2. The process according to claim 1, wherein the basic layer (21) is in the form of a continuous sheet or band.

3. The process according to claim 2, wherein the basic layer (21) and the intermediate mass (31) are advanced with continuous advancing motion.

4. The process according to claim 1, wherein the portions or laps (41) defining the intermediate mass (31) are defined by portions or strips, preferably longitudinal portions or strips, more preferably longitudinal and adjacent portions or strips, of the basic layer (21).

5. The process according to claim 1, wherein the intermediate mass (31) is in the form of a continuous or elongate body.

6. The process according to claim 1, wherein the folding step comprises extending one or more portions or laps (41) of the basic layer (21) perpendicularly to a plane (L-T) defined by the basic layer (21) in a plane condition.

7. The process according to claim 1, wherein the step of folding the basic layer (21) comprises making in the selfsame basic layer (21) one or more pleat portions, or pairs of laps (41, 41), that define respective layers of the intermediate mass (31).

8. The process according to claim 1, wherein the step of folding the basic layer (21) comprises performing one or more, preferably a plurality of, folding steps in succession on the same basic layer (21).

9. The process according to claim 8, wherein each folding step comprises making at least one pleat portion, or pair of laps (41, 41).

10. The process according to claim 1, wherein a height of a respective portion or lap (41) of said basic layer (21) defines a height (h′) of the intermediate mass (31).

11. The process according to claim 1, wherein it comprises bringing closer together the portions or laps (41) obtained from said basic layer (21) to define said intermediate mass (31).

12. The process according to claim 1, wherein it comprises compressing one against the other the portions or laps (41) obtained from said basic layer (21) to define the intermediate mass (31).

13. The process according to claim 12, wherein compression one against the other of the portions or laps (41) obtained from the basic layer (21) is exerted along a direction of stress parallel to a plane (L-T) defined by the basic layer (21) in a plane condition.

14. The process according to claim 1, wherein the solid form (11) is obtained from the intermediate mass (31) by cutting the intermediate mass (31).

15. The process according to claim 1, wherein the solid form (11) is obtained from the intermediate mass (31) by compression moulding of the intermediate mass (31) in a respective mould.

16. An apparatus (10) for making a solid form (11) of material, said material being a therapeutic or pharmaceutical product, a dietary supplement, and/or a food or edible product, wherein it comprises folding means (16, 116) which, starting from at least one basic layer (21) of the material in the form of a sheet or band, fold the at least one basic layer (21) so as to make a plurality of portions or laps (41); means (30; 40) which bring the plurality of portions or laps closer together to define an intermediate mass (31); and means (40) adapted to obtain a solid form (11) from the intermediate mass (31) by separation.

17. The apparatus according to claim 16, wherein the folding means (16, 116) are adapted to pleat the at least one respective basic layer (21) to make the intermediate mass (31) of the material.

18. The apparatus according to claim 16, wherein it comprises feeding means (20) for feeding the at least one respective basic layer (21) of material.

19. The apparatus according to claim 16, wherein the basic layer (21) of material is in the form of a respective continuous sheet or band.

20. The apparatus according to claim 16, wherein the intermediate mass (31) is in the form of a continuous, elongate body.

21. The apparatus according to claim 16, wherein the means (16, 116) for folding the basic layer (21) make in the selfsame basic layer (21) one or more pleat portions, or pairs of laps (41, 41), that define respective layers of the intermediate mass (31).

22. The apparatus according to claim 16, wherein the means (16, 116) for folding the basic layer (21) comprise engagement means (17a, 19a; 117a, 119a) for the basic layer (21) which comprise respective first protruding means (17a, 117a) and respective second protruding means (19a, 119a), the first and second protruding means (17a, 19a; 117a, 119a) extending longitudinally according to a respective profile along an advancing direction between an engagement position, where they engage opposite longitudinal faces (23, 25) of the basic layer (21) when the folding of a respective pleat portion (41, 41) begins, and a transversal close together position where they are alongside each other and portions or laps (41, 41) of the material are interposed in between them to define a corresponding pleat portion (41, 41).

23. The apparatus according to claim 22, wherein the first and second protruding means comprise protrusions (17a, 19a; 117a, 119a) for engaging the basic layer (21), and having a transversal cross section with a wedge-shaped, or triangular, profile converging towards the basic layer (21).

24. The apparatus according to claim 16, wherein it comprises one or more, preferably a plurality of, folding sections (16a, 16b, 16c, 16d, 16e, 16f, 16g, 16z; 116a, 116b, 116c, 116d, 116e, 116f, 116g, 116z), each having a first and a second folding member or element (17o, 19o, 117o, 119o) which engage opposite longitudinal faces (23, 25) of the basic layer (21) and mutually cooperate to make one or more corresponding pleat portions (41, 41).

25. The apparatus according to claim 16, wherein the folding means (16, 116) comprise a first and a second body (17, 19) located at opposite longitudinal faces (23, 25) of the basic layer (21) and which have corresponding protrusions (17a, 19a) extending along an advancing direction of the basic layer (21).

26. The apparatus according to claim 25, wherein said first and second bodies (17, 19) define in between them a plurality of longitudinal and parallel channels for folding the basic layer (21).

27. The apparatus according to claim 16, wherein the folding means (116) are in the form of movable means, in particular movable along an advancing direction of the basic layer (21).

28. The apparatus according to claim 27, wherein the folding means (116) are movable in rotation about an axis parallel to a plane (L-T) defined by the basic layer (21) in the plane condition, that is, parallel to a transversal direction (T).

29. The apparatus according to claim 16, wherein the means (18) for bringing the plurality of portions or laps (41) closer together comprise compression means (30, 40) adapted to compress the portions or laps (41) of the basic layer (21).

30. The apparatus according to claim 29, wherein primary compression means (30) for the portions or laps of the basic layer (21) are provided, in particular to define the intermediate mass (31).

31. The apparatus according to claim 30, wherein the primary compression means (30) comprise a pair of opposed rolls (30a, 30b) for engaging and compressing interposed portions or laps (41) of the basic layer (21), the rolls (30a, 30b) being rotatable about respective axes perpendicular to a plane (L-T) defined by the basic layer (21) in the plane condition.

32. The apparatus according to claim 29, wherein final compression means (40) are provided for the portions or laps (41) of the basic layer (21).

33. The apparatus according to claim 16, wherein the means (40) adapted to obtain a solid form (11) from the intermediate mass (31) by separation are in the form of cutting means, in particular adapted to make a transversal cut in the intermediate mass (31).

34. The apparatus according to claim 16, wherein the means (40) adapted to obtain a solid form (11) from the intermediate mass (31) by separation are in the form of compression moulding means (40) for the intermediate mass (31).

35. The apparatus according to claim 34, wherein the compression moulding means (40) define final compression means for the intermediate mass (31).

36. The apparatus according to claim 34, wherein the compression moulding means (40) comprise a first and second opposed moulding parts (141, 42) carried on a pair of opposed wheels (40a, 40b) which engage the intermediate mass (31) of material, the wheels (40a, 40b) being rotatable about respective axes perpendicular to a plane (L-T) defined by the basic layer (21) in the plane condition and having a shaped peripheral surface defining a plurality of contiguous and successive half-moulds (141, 42).

37. The apparatus according to claim 16, wherein it comprises pulling means (30, 40) for the intermediate mass (31) and/or the basic layer (21).

38. The apparatus according to claim 37, wherein the pulling means (30, 40) are located downstream of the folding means (16, 116) for the basic layer (21) and are adapted to advance the basic layer (21) and the intermediate mass (31) with continuous advancing motion.

39. The apparatus according to claim 16, wherein it comprises a compensating buffer (22) for storing a reserve of basic layer (21) being fed to the folding means (16; 116) downstream thereof.

40. The apparatus according to claim 16, wherein it comprises, at the folding means (16; 116), means (50) adapted to define a guide plane in which the basic layer (21) lies.

41. The apparatus according to claim 16, wherein it comprises means, in particular the folding means, which are adapted to provide the intermediate mass (31) with respective laps or layers (41, 41) which differ in height from each other, in particular a maximum height (ha) in the central zone (131a) and a minimum height (hb), in the opposed lateral zones (131b, 131b) of the intermediate mass (31), and possibly a height (hc), between the maximum height (ha) and the minimum height (hb) in the intermediate zones (131c, 131c) between the central zone (131a) and the lateral zones (131b, 131b), the height of the intermediate zones decreasing progressively from the central zone (131a) towards the lateral zones (131b, 131b) of the intermediate mass (31).

42. The apparatus according to claim 16, wherein respective pairs of opposed shaping members or elements (117o, 119o) are perpendicularly adjustable independently of each other.

43. The apparatus according to claim 16, wherein the pairs of opposed shaping members or elements (117o, 119o) are perpendicularly adjustable, or positionable, in such a way that the first pair of opposed shaping elements, or members, (116a) is spaced at a minimum distance from each other and the last pair of opposed shaping elements, or members, (116z) is spaced at a maximum distance from each other, each of the intermediate pairs of opposed shaping elements, or members (116b, 116c, 116d, 116e, 116f, 116g) being preferably spaced at a respective distance which is between the minimum distance and the maximum distance and which preferably increases progressively starting from the minimum distance of the first pair of opposed shaping elements, or members (116a) towards the last pair opposed shaping elements, or members (116z).

44. The apparatus according to claim 16, wherein it comprises means for adjusting the compression applied by the means (30) for the primary compression of the intermediate mass (31).

45. The apparatus according to claim 16, wherein it comprises means for adjusting the spacing between the rolls (30a, 30b) for the engagement and compression of the intermediate mass (31).

46. The apparatus according to claim 44, wherein it comprises an engagement and compression roll (30b) which is movable, being in particular movable along a transversal direction (T) relative to an engagement and compression roll (30a) which is fixed.

47. The apparatus according to claim 46, wherein the movable engagement and compression roll (30b) is supported by a respective arm (131) which is pivoted at one end (131a) and has a free opposite end (131b) which is movable along the transversal direction “T” slidably on corresponding guide means and which is operatively connected to respective drive means, or motor (132).

48. The apparatus according to claim 44, wherein it comprises means for detecting a force exerted by the movable engagement and compression roll (30b) on the fixed engagement and compression roll (30a), the detecting means providing a signal used by corresponding control means to control the spacing between the movable engagement and compression roll (30b) and the fixed engagement and compression roll (30a), that is, to suitably adjust the state of compression of the intermediate mass (31).

49. The apparatus according to claim 16, wherein it comprises means for regulating the temperature of the intermediate mass (31).

50. The apparatus according to claim 49, wherein it comprises means for detecting the temperature of the intermediate mass (31), connected to respective control means, and means for thermally conditioning the intermediate mass (31), driven by the control means.

51. The apparatus according to claim 49, wherein the means for regulating the temperature of the intermediate mass (31) are located at the means (30) for the aggregation or precompression of the intermediate mass (31).

52. The apparatus according to claim 49, wherein it comprises means for detecting the temperature of the respective roller (30a, 30b) of the aggregation or precompression means (30).

53. The apparatus according to claim 49, wherein the means for regulating the temperature of the material are designed to heat the respective roller (30a, 30b) of the aggregation or precompression means (30).

54. The apparatus according to claim 49, wherein it comprises at least one conduit inside the body of the roller (30a, and/or 30b), the conduit being designed to have a heating fluid flowing through it.

55. The apparatus according to claim 16, wherein it comprises means for expelling the solid form (11) from the moulding means (40), in particular from the respective forming mould.

56. The apparatus according to claim 55, wherein the expulsion means comprise pushing means by which the solid form (11) is expelled from the respective cavity of the mould or half mould (40).

57. The apparatus according to claim 55, wherein the inner end wall (43e) of the respective cavity (42) of the forming mould or half mould (40a, 40b) is movable wholly or partly between a retracted moulding position and an extended position for releasing or expelling the solid form (11).

58. The apparatus according to claim 55, wherein the inner end wall (43e) of the cavity (42) of the mould or half mould of the moulding means is connected to, or integral with, a block, or slider, (143) that mounts a respective roller, or similar element, (144), which is slidable on a corresponding cam (145) which is designed to control the movement, in particular the radial movement, of the inner end wall (43e) between the retracted moulding position and the extended position for releasing or expelling the solid form (11).

59. The apparatus according to claim 58, wherein the cam (145) has a long main circumferential stretch (145a), largely circular in shape, by which the inner end wall (43e) of the cavity (42) is placed in the retracted moulding position, and a short circumferential stretch (145b), which is radially outside the main stretch and by which the inner end wall (43e) of the cavity (42) is placed in the extended position for releasing or expelling the solid form (11).

60. A solid form (11) of respective material, the material being a therapeutic or pharmaceutical product, a dietary supplement, and/or a food or edible product, wherein it is obtained by means of a process according to claim 1.
